(19) United States
(12) Patent Application Publication
(10) Pub. No.: US 2025/0312672 A1
(43) Pub. Date: Oct. 9, 2025

(54) BASKETBALL FORM TRAINING SYSTEM

(71) Applicant: Airborne Athletics, Inc., Bloomington, MN (US)

(72) Inventors: Justin Royer, Maricopa, AZ (US); Jefferson William Mason, Maple Grove, MN (US); Julia M. Ploof, Minneapolis, MN (US); Lucas Pillman, Burnsville, MN (US); Eric David Drommerhausen, Maplewood, MN (US); Eric Pollpeter, Minneapolis, MN (US); Douglas Brad Campbell, Loretto, MN (US)

(21) Appl. No.: 19/089,923

(22) Filed: Mar. 25, 2025

Related U.S. Application Data
(60) Provisional application No. 63/574,581, filed on Apr. 4, 2024.
(60) Provisional application No. 63/654,426, filed on May 31, 2024.

(51) Int. Cl. A63B 69/00; A63B 24/00; A63B 69/40; A63B 71/06

(52) U.S. Cl. CPC A63B 69/0071; A63B 24/0006; A63B 24/0021; A63B 24/0062; A63B 24/0075; A63B 69/40; A63B 2024/0068; A63B 71/0622; A63B 2071/063; A63B 2214/00; A63B 2220/805; A63B 2220/806; A63B 2220/89; A63B 2230/62

(57) ABSTRACT

A sports training system including a sport training machine, a sensor, and one or more processors coupled to one or more computer-readable storage media having instructions stored thereon which, when executed by the one or more processors, cause the one or more processors to perform operations including obtaining, from the sensor, sensor data including a person performing a player movement, extracting, from the sensor data, action data including multiple points including respective locations of biometric data points corresponding to the person appearing in the multiple frames and tracking the player movement, identifying, from the action data, two or more phases of the player movement each including a proper subset of the multiple points tracking a timing and movement of the phase of the player movement, and generating, from the extracted action data, a movement profile for the player movement including the two or more phases.

### CROSS-REFERENCE TO RELATED APPLICATIONS

This application claims the benefit of priority to U.S. Provisional Application No. 63/574,581, filed on Apr. 4, 2024, and to U.S. Provisional Application No. 63/654,426, filed on May 31, 2024, the contents of which are both hereby incorporated by reference.

### BACKGROUND

This disclosure relates generally to sports training, and in particular to basketball return systems with a user interface.

Successful shooting of a basketball can be affected by a number of factors, including a player's form or technique in shooting. In some cases, poor form or technique may have less effect when the player is taking uncontested shots from similar distances but may limit the player's ability to score in game conditions when the player is guarded by another player and often must attempt shots from varying positions on the court having varying distances from the basketball goal.

As players advance in skill and experience, they are often confronted with the realization that the speed of the game gets “faster,” and that he or she will need to consistently score under increasing pressure and from various positions on the court. Continuing to practice under conditions that do not effectively simulate the level of movement and consistency required of the shooter and the variety of shot locations frequently encountered in game conditions can result in some improvement in the player's shooting but may ultimately limit the player's success as the player rises through the levels of play from, e.g., junior varsity to varsity, from high school varsity to college, and from college to professional basketball.

### SUMMARY

This specification describes technologies for providing form training using a basketball training system to provide feedback to players that are practicing using the system.

These technologies generally involve a basketball training system that uses collected sensor data to determine action profiles for actions performed by players that are practicing using the system. With these action profiles, the system is able to identify player form relative to ideal form and/or self-consistent form and provide feedback to the player, e.g., provide suggested drills to correct form deviation. This can allow for real-time feedback to the player, summary and statistical information about the practice session, accurate form tracking, and other advantages compared to environments without this technology.

The basketball training system can capture sensor data from an external or integrated sensor, e.g., video via a camera on or attached to a basketball training machine, or from a sensor that is separate from a basketball training machine. For example, the basketball training system can use computer vision on a picture or video captured by a user's camera. The video from the user's camera (e.g., a user's mobile device) can be uploaded to, sent to, or otherwise made available to the basketball training system for computer vision.

The system captures sensor data of a player performing multiple iterations of an action (e.g., a basketball drill or standard) and extracts, from the sensor data, action data representative of the action at multiple points, e.g., biometric data of the person as well as object data of an object captured by the sensor data (e.g., the basketball). For example, the system can extract biomechanical points representative of locations of features of the person, e.g., hand location, foot location, as well as define relative locations of biomechanical points between features of the person, e.g., shoulder distance, hip distance, location of hand to face, location of hips over feet, etc.

The system can capture sensor data from two or more sensors of the player performing an action. The two or more sensors can be the same type of sensor, e.g., two or more cameras, two or more mmWave sensors, or the like. In some instances, the two or more sensors can be different types of sensors, where the system captures respective types of sensor data from the two or more different types of sensors of the player performing the action and merges the multiple streams of sensor data to generate an action profile for the action.

The system can generate, from the extracted biometric data and object data, an action profile (e.g., shot profile) for the action, where the action profile tracks (i) timing and (ii) mechanics of the action using multiple points of the extracted biometric and object data. The system can determine, from the action profile, multiple different phases of the action profile, each phase corresponding to a sub-action and represented in the action profile by a set of points of the biometric data and object data. Each of the phases can track a different, respective sub-set of the multiple points of the action data.

At least one phase can overlap with another different phase in time, for example, a shot motion can overlap with a shot position, shot path, and follow through. Different metrics can be used to quantify each of the phases, and a threshold range of acceptable timing and/or mechanics can be used for each respective phase, i.e., a first phase may have a larger range of acceptable timing and/or mechanics than a different phase.

Action profiles capturing respective actions by the person can be aligned using a respective reference point in each action profile. For example, a reference point can be a lowest (e.g., along a y-axis) of the shot pocket. The system can average of the action profiles in a sequence of captured action profiles (e.g., during a drill including multiple shots taken) and evaluate the averaged shot profile to determine if one or more of the phases of the averaged action profile is outside a threshold deviation.

Video data can include videos of the action captured from two or more perspectives (e.g., profile and face-on). In some implementations, computer vision and a trained model can be used to infer additional perspectives from video data capturing one or more perspectives. E.g., a model can infer a profile view of the action using a face-on perspective video data of the action.

Video data can be enriched with additional sensor data collected from one or more other types of sensors in addition to the video data captured using a camera. For example, mmWave radar data can be used to enrich video data to provide metadata for the action captured using the camera.

The system includes a user interface to guide the player through collecting the shot action sensor data, e.g., a number of shots from each perspective. The system can provide feedback on the player's form through the user interface and can include one or more training regimens to assist the player in adjusting form based on an identified deviation of the action profile from an ideal and/or self-consistent profile.

In one example, a basketball training system includes a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In general, one innovative aspect of the subject matter described in this specification can be embodied in a basketball training system including a basketball training machine, a sensor, and one or more processors coupled to one or more computer-readable storage media having instructions stored thereon which, when executed by the one or more processors, cause the one or more processors to perform operations. The operations include obtaining, from the sensor, sensor that include a person performing a shot action, extracting, from the sensor, action data including multiple points tracking the shot action, where the multiple points track respective locations of biometric data points corresponding to the person appearing in the sensor and tracking the shot action. The operations include identifying, from the action data, two or more phases of the shot action, where each of the two or more phases of the shot action include a proper subset of the multiple points tracking a timing and movement of the phase of the shot action, and generating, from the extracted action data, a shot profile for the shot action including the two or more phases.

The foregoing and other embodiments can each optionally include one or more of the following features, alone or in combination. In particular, one embodiment includes all the following features in combination. In some implementations, the operations of the system further includes evaluating the shot profile for the shot action, where the evaluating includes providing, for each of the two or more phases of the shot action and to a corresponding trained machine learning model of multiple trained machine learning models, the proper subset of the multiple points tracking the timing and movement of the phase of the shot action, and obtaining, for each of the two or more phases of the shot action, phase metrics for the phase.

In some implementations, the trained machine learning models are each trained on a respective, different data set corresponding to a phase of the two or more phases of a shot profile for the action shot.

In some implementations, the operation of the system further include evaluating, from the phase metrics of each phase of the two or more phases of the shot profile, the shot profile against an ideal shot profile, determining, from the evaluation, a deviation of the shot profile from the ideal shot profile for a phase of the two or more phases is larger than a threshold deviation, and providing, to the person, a training alert. Providing the training alert can further include determining, for the phase having a deviation larger than the threshold deviation, a training regimen specific to the phase and different than a training regimen for each other phase of the two or more phases, and providing, to the person, the training alert.

In some implementations, determining the deviation of the shot profile from the ideal shot profile for the phase includes determining, a type of deviation of multiple types of deviations for the phase, and where determining the training regimen specific to the phase includes determining the training regimen of multiple training regimens that is specific to the type of deviation of the multiple types deviations for the phase.

In some implementations, providing the training alert includes providing, for presentation in a user interface, a visual representation indicating the phase of the two or more phases for which the deviation of the shot profile from the ideal shot profile is larger than the threshold deviation, and providing, for presentation in the user interface, a selectable control operable to initiate the training regimen specific to the phase.

In some implementations, obtaining the sensor includes obtaining the sensor data including the person performing the shot action two or more times, and where generating the shot profile includes generating a respective shot profile for each of the two or more performed shot actions by the person.

In some implementations, obtaining the sensor data further includes identifying, for each of the respective shot profiles, a corresponding reference point of the shot profile, and aligning the shot profiles for the two or more performed shot actions using the respective reference points of the shot profiles.

In some implementations, the operation of the system further includes generating, from each of the two or more shot actions, an average shot profile for the shot action for the person, evaluating, from the average shot profile against an ideal shot profile, determining, from the evaluation, a deviation of the average shot profile from the ideal shot profile for a phase of the two or more phases is larger than a threshold deviation, and providing, to the person, a training alert.

In some implementations, the action data including the multiple points tracking the shot action further includes tracking respective locations of an object appearing in the multiple frames, and where tracking the respective locations of the object includes tracking the object relative to the biometric data points corresponding to the person in the multiple frames.

In some implementations, each phase of the two or more phases is defined by coordinate points and vectors for the points through a duration of the phase of the action shot.

In some implementations, the two or more phases of the shot action include position, shot pocket, lift, shot path, follow through, and landing.

In some implementations, at least one phase of the two or more phases overlaps in timing with at least one other phase of the two or more phases during the shot action.

In some implementations, the sensor is an image sensor, and the sensor data includes video data including multiple frames including the person performing the shot action. In some implementations, obtaining, from the image sensor, the video data includes providing, for presentation in a user interface, a first visual indication of a first position to arrange the image sensor with respect to the person performing the shot action to capture the video data, receiving, from the image sensor, the video data, determining, from the video data, that a threshold video data capturing the shot action from the first position is met, and providing, for presentation in the user interface, a second visual indication of a completion of capture of the video data from the first position. Obtaining the video data can further include providing, for presentation in the user interface, a third visual indication of a second position to arrange the image sensor with respect to the person performing the shot action to capture the video data, where the second position is at a different angle with respect to the basketball training machine from the first position, receiving, from the image sensor, the video data, determining, from the video data, that a threshold video data capturing the shot action from the second position is met, and providing, for presentation in the user interface, a fourth visual indication of a completion of capture of the video data from the second position.

In some implementations, the first position is a side view of the person performing the shot action, and where the second position is a face-on view of the person performing the shot action.

In some implementations, determining, from the video data, that the threshold video data capturing the shot action from the first position and the second position is met includes determining that a threshold number of shot actions are captured in the multiple frames of the video data from the respective first position and the second position. The threshold number of shot actions can be different between the first position and the second position.

In some implementations, obtaining, from the image sensor, the video data includes providing, to a trained machine learning model, the video data capturing the shot action from the first position, and obtaining, from the trained machine learning model, extrapolated synthetic video data representing the shot action from a second, virtual position.

In some implementations, the shot action includes the action of receiving, by the person and from a delivery device of the basketball training machine, a basketball, and shooting, by the person and to a receiving hoop of the basketball training machine, the basketball.

In some implementations, the operation of the system further includes obtaining, from the basketball training machine and for the shot action, shot completion feedback, and generating, from the shot profile and the shot completion feedback; an enriched shot profile for the shot action.

In some implementations, the sensor is configured to capture sensor data in a non-visible electromagnetic spectrum frequency band. The sensor can be configured to capture sensor data in one of radio frequency bands, microwave frequency bands, acoustic frequency bands, and infrared frequency bands. The sensor data can include point cloud data.

In general, another innovative aspect of the invention includes a sports training system including a sport training machine, a sensor, and one or more processors coupled to one or more computer-readable storage media having instructions stored thereon which, when executed by the one or more processors, cause the one or more processors to perform operations including obtaining, from the sensor, sensor data including a person performing a player movement, extracting, from the sensor data, action data including multiple points tracking the player movement, where the multiple points track respective locations of biometric data points corresponding to the person appearing in the multiple frames and tracking the player movement, identifying, from the action data, two or more phases of the player movement, where each of the two or more phases of the player movement include a proper subset of the multiple points tracking a timing and movement of the phase of the player movement, and generating, from the extracted action data, a movement profile for the player movement including the two or more phases.

The foregoing and other embodiments can each optionally include one or more of the following features, alone or in combination. In particular, one embodiment includes all the following features in combination. In some implementations, the sensor includes a first sensor to obtain first second data and the system further includes a second sensor, where the operations further include obtaining, from the second sensor, second sensor data including the person performing the player movement, extracting, from the second sensor data, second action data including a second multiple points tracking the player movement, where the second multiple points track respective locations of biometric data points corresponding to the person captured in the second sensor data and tracking the player movement.

In some implementations, the operations further include identifying, from the action data and the second action data, two or more phases of the player movement, where each of the two or more phases of the player movement include a proper subset selected from the multiple points and the second multiple points tracking a timing and movement of the phase of the player movement, and generating, from the extracted action data and second action data, the movement profile for the player movement including the two or more phases.

In some implementations, the first sensor includes an image sensor and is configured to capture video data and the second sensor is configured to capture the second sensor data in one of radio frequency bands, microwave frequency bands, acoustic frequency bands, and infrared frequency bands.

In some implementations, the proper subset is selected from both of the multiple points and the second multiple points.

In general, another innovative aspect of the invention includes a sports training method including providing, by an emitter, a source signal including a non-visible wavelength, collecting, from a sensor and from reflections of the source signal, sensor data including a person performing a player movement, extracting, from the sensor data, action data including multiple points tracking the player movement, where the multiple points track respective locations of biometric data points corresponding to the person appearing in the multiple frames and tracking the player movement, identifying, from the action data, two or more phases of the player movement, where each of the two or more phases of the player movement include a proper subset of the multiple points tracking a timing and movement of the phase of the player movement, and generating, from the extracted action data, a movement profile for the player movement including the two or more phases.

The technology described in this specification can be implemented so as to realize one or more of the following advantages. The basketball training system can allow a user to practice their basketball skills in an efficient and effective manner. For example, the basketball training system can provide basketball players the ability improve their basketball skills with an increased amount of practice reps in each practice session.

Further, the basketball training system provides feedback regarding the performance of the basketball player to ensure that the player is practicing properly to ensure their practice time is spent curating habits and skills that are correct and will facilitate the player's improvement. By representing different phases of the shot action mathematically, the system is able to deconstruct and quantify a user's action shot into the different phases and automatically determine aspects of the user's form that deviate from an ideal form and/or a self-consistent form, which may be non-obvious even to a skilled coach or trainer. The system can track a user's shot over multiple shots in a single session or a user's shot over multiple sessions such that the system can detect and correct issues with consistency of form that may not be possible for a coach or trainer to track for multiple players over time and/or over a large volume of shots.

The basketball training system provides real-time performance feedback including shot completion feedback (e.g., make, miss, miss bias, etc.) to a user that allows a user to learn why a shot was made and/or why a shot was missed based on the basketball training system's ability to analyze the form of the user, e.g., which phases of the shot action are likely leading to the shot made/missed outcome and provide feedback regarding the user's form for made and missed shots. In some implementations, the system can integrate additional shot action data related to spatial location of missed shots with respect to the basketball rim to generate insight of which aspects of a user's shot action to miss bias of the missed shots and generate, from the insight, targeted training to correct for the problematic aspects of the user's shot action.

In some implementations, the synthesized action shot data including shot biomechanics and shot completion feedback (i.e., make/miss, miss bias, etc.) can be used to train a machine learning predictive model such that the user's shot biomechanics are provided as input to the trained machine learning model and a predictions related to a shot outcome is received as output from the trained machine learning model. The predictive model can further provide non-obvious insight into specific phases of the user's shot action that are likely leading to shot misses, which can be used to generate customized, specific workouts to target and correct the issues.

The technology described in this specification can allow users to virtually compete with other users to see who can align their form closest with an ideal form and/or who can be most consistent with their form (e.g., as compared with themselves).

In some implementations, the basketball training machine including a sensing subsystem including a non-visible wavelength source, e.g., emitter, and a sensor, e.g., detector, configured to detect reflections of the source from the training environment surrounding the basketball training machine. In such instances, the wavelengths emitted by the source can be, for example, millimeter wave (mmWave), LIDAR, microwave radar, or other non-visible wavelength bands of the electromagnetic spectrum. An operating wavelength band of the sensing subsystem can be selected in part using various factors, for example, accuracy, precision, sensing speed, resolution, and range of detection. The operating wavelength band can be selected, for example, based on a resolution requirement for classifying the different aspects of the shot action.

Non-visible wavelength-based sensing subsystems can offer increased robustness to environmental factors over visible-based sensing, e.g., cameras. For example, non-visible based sensing can be robust to lighting conditions, weather interference (e.g., rain), contaminants such as dust or other particles, and the like.

In some implementations, non-visible frequency-based sensing can be incorporated in addition to, or alternatively to, camera-based sensing in the basketball training system. Non-visible sensing data, e.g., point cloud data, can be used instead of or to enrich visible-wavelength camera-based imaging data. In some examples, the system can include two or more types of sensors collecting respective sensor data. For example, video data and mmWave radar data. In such cases, the video data capturing an action can be enriched using the mmWave radar data to provide enhanced capture of the player and/or the ball movement before, during, and after the action is performed. In some examples, non-visible wavelength sensing subsystems can increase privacy of users by limiting feature resolution of the collected sensor data, e.g., not including facial information.

In some examples, a mmWave-based sensing subsystem can provide high-precision, low latency object (e.g., player and ball) sensing capabilities. A mmWave-based sensing subsystem can provide added robustness, e.g., over alternative radar technologies, to environmental interference. In some examples, mmWave-based sensing can additionally include Doppler functionality, e.g., measuring speed/velocity of a player before, during, and after the shot action.

For example, a mmWave-based sensing subsystem can offer increased robustness to variations in lighting conditions, e.g., low light, dust/particles, and weather conditions such as rain or fog (if in an outdoor environment). In cases where mmWave-based sensing is used, the collected sensor data, e.g., raw point cloud data, can be processed to protect the privacy of the players captured in the sensor data. The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a side view of a basketball training machine that includes a ball collection system and a ball delivery system that delivers basketballs to selected ball delivery locations included in a workout program defined via a graphical user interface.

FIG. 2 is a block diagram of an example operating environment of a basketball training system.

FIG. 3 is a flow diagram of an example process of the basketball training system.

FIG. 4 is a flow diagram of another example process of the basketball training system.

FIG. 5 is a flow diagram of another example process of the basketball training system.

FIG. 6 is a flow diagram of another example process of the basketball training system.

FIG. 7 is a schematic of an example of a biomechanical pose detection schema.

FIG. 8 is an example plot of shot profiles.

FIG. 9 is an example plot of phases of a shot profile.

FIG. 10 is an example of a user interface of the basketball training system.

FIG. 11 is an example of a user interface of the basketball training system.

FIG. 12 is a flow diagram of an example process of the basketball training system.

Like reference numbers and designations in the various drawings indicate like elements.

### DETAILED DESCRIPTION

A basketball training system uses computer vision to perform form training for players that are practicing using the system. By collecting and processing player video data of a player performing an action, e.g., during a static or dynamic shooting action, while dribbling, or the like, the system can capture biomechanical information of the player. The system is able to identify player form deviation with respect to an ideal form and/or self-consistent form, correlate the deviations with corrective drills and standards, and provide feedback to the player. This can allow for real-time feedback and corrective action for the player, summary and statistical information about the practice session, and accurate form tracking and correction.

The basketball training system can capture sensor data, e.g., video, via one or more sensors on or attached to a basketball training machine or can receive captured sensor data from one or more sensors that are separate from the basketball training machine. For example, the computer vision system can receive video data captured by a mobile computing device of a user (e.g., a cellular phone or tablet with a camera). An application on the device can record or transmit video, or data from the video, to the basketball training system, or the application on the device itself may perform one or more types of computer-vision analysis.

The system can integrate mathematical representations of a shot action, e.g., a basketball shot, and identify a subset of points of the biomechanical data extracted from the video data captured of the shot action to represent the different biomechanical phases of the shot action. The system can assess the shot action over multiple performed shots, e.g., a sequence of shots, to determine consistency of the player with respect to an ideal form and/or self-consistent form

This can advantageously allow for more accurate, dynamic, and proactive basketball training than otherwise possible with other basketball training systems that lack these features. For example, a basketball training system that does not have this type of computer vision analysis would be unable to observe user's form when they make or miss a shot, meaning less useful feedback could be provided-only that the player missed a shot, and not an analysis of why or automated recommendations for drills specific to the particular form of a given player when missing a shot. For example, two players may both have identical free throw percentages (e.g., 66%). With pose information from the computer vision, the basketball training system may determine that one player has a tendency to flair their elbows when they miss, and the other play tends to release the ball too low when they miss. With this information, the two players can be provided with recommendations for different drills-one to focus on elbow placement and the other on ball-release point.

### Basketball Training System

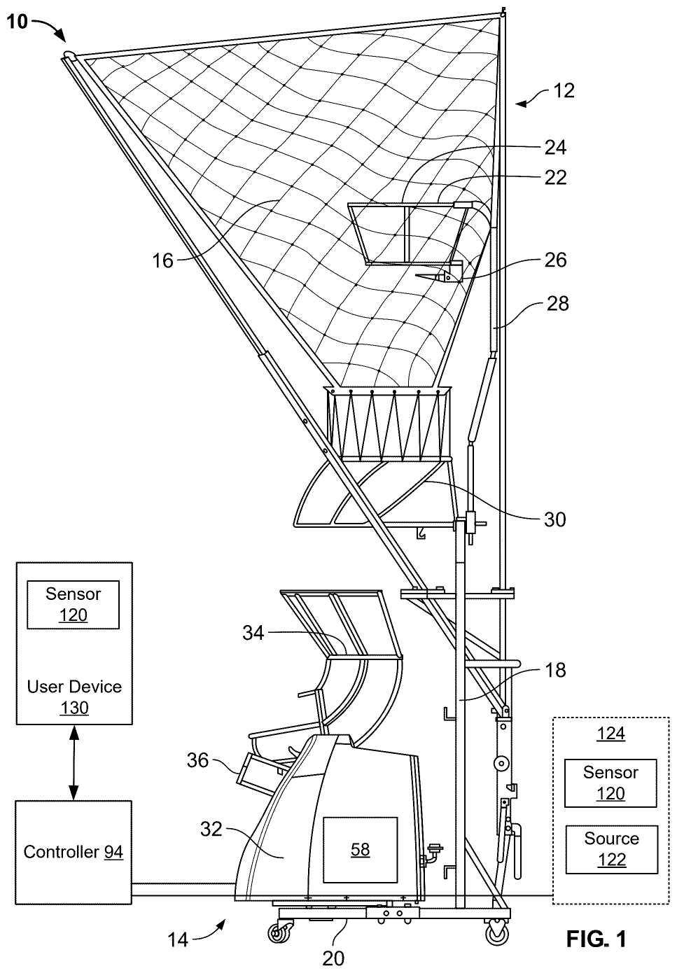

FIG. 1 shows a side view of basketball training machine 10. Basketball training machine 10 includes two main systems, ball collection system 12 and ball delivery system 14. Further description of basketball training machine 10 can be found in currently-pending patent application Ser. No. 18/100,814, filed on Jan. 24, 2023, and entitled BASKETBALL TRAINING SYSTEM WITH COMPUTER VISION FUNCTIONALITY.

While described herein with respect to a basketball training machine 10 of a basketball training system, it should be understood that aspects of basketball training machine 10 can be applied to other ball sports as well. For instance, basketball training machine 10 can deliver volleyballs, soccer balls, or other types of balls for training purposes for such other sports. As such, basketball training machine 10 can be considered, in some examples, as a ball sports training machine.

Ball collection system 12 includes net 16, net frame 18, base 20, shots made counter 22 (which, in this embodiment, includes made shots funnel 24, shots made sensor 26, and counter support frame 28), and upper ball feeder 30. When machine 10 is used for shooting practice, net 16 is positioned in front of a basketball backboard (not shown) so that the basketball hoop and net (not shown) are immediately above shots made counter 22. The size of net 16 is large enough so that missed shots (which do not go through the basketball hoop and net and through shots made counter 22) will still be collected by net 16 and funneled down to upper ball feeder 30.

Ball delivery system 14 includes ball delivery machine 32, main ball feeder 34, and ball ready holder 36. The inlet of main ball feeder 34 is positioned immediately below the outlet of upper ball feeder 30. Ball delivery machine 32 is pivotally mounted on base 20. Ball delivery machine 32 is pivotable about an axis that is aligned with the inlet of main ball feeder 34 and the outlet of upper ball feeder 30. Balls drop out of upper ball feeder 30 into main ball feeder 34. Balls are delivered one at a time from main ball feeder 34 into ball ready holder 36 at the front of ball delivery machine 32. A launch arm (not shown) launches the basketball out of holder 36 to a location on the floor where the player catches the ball and shoots. The location on the floor where the ball is delivered can be changed by pivoting machine 32 with respect to base 20.

Basketball training machine 10 includes a control system, e.g., controller 94, in data communication with the ball delivery system 14 and ball collection system 12. Controller 94 is a processor-based controller that coordinates the operation of components of the control system of the basketball training machine 10. Controller 94 includes one or more processors and computer-readable memory encoded with instructions that, when executed by the one or more processors, cause controller 94 to operate in accordance with techniques described herein. Examples of one or more processors of controller 94 can include any one or more of a microprocessor, a controller, a digital signal processor (DSP), an application specific integrated circuit (ASIC), a field-programmable gate array (FPGA), or other equivalent discrete or integrated logic circuitry.

Computer-readable memory of controller 94 can be configured to store information within controller 94 during operation. Computer-readable memory of controller 94, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, the computer-readable memory is a temporary memory, meaning that a primary purpose of the computer-readable memory is not long-term storage. Computer-readable memory, in some examples, includes volatile memory that does not maintain stored contents when electrical power to controller 94 is removed. Examples of volatile memories can include random access memories (RAM), dynamic random access memories (DRAM), static random access memories (SRAM), and other forms of volatile memories. In some examples, computer-readable memory of controller 94 is used to store program instructions for execution by the one or more processors of controller 94. For instance, computer-readable memory of controller 94, in some examples, is used by software or applications running on controller 94 to temporarily store information during program execution.

Computer-readable memory of controller 94, in some examples, also includes one or more computer-readable storage media that can be configured to store larger amounts of information than volatile memory. In some examples, computer-readable memory of controller 94 includes non-volatile storage elements. Examples of such non-volatile storage elements can include magnetic hard discs, optical discs, floppy discs, flash memories, or forms of electrically programmable memories (EPROM) or electrically erasable and programmable (EEPROM) memories.

Controller 94 utilizes communication device(s) to communicate with external devices via one or more wired or wireless communication networks, or both. Communication device(s) can include any one or more communication devices, such as network interface cards (e.g., Ethernet cards), optical transceivers, radio frequency transceivers, Bluetooth transceivers, 3G or 4G transceivers, and WiFi radio computing devices.

The control system is configured to provide control commands to components of the basketball training machine 10. For example, control system is configured to provide control commands to ball delivery machine 32 to cause ball delivery machine 32 to launch basketballs in directions based upon the selected ball delivery locations.

In some implementations, the control system is configured to provide control commands to ball delivery machine 32 to cause ball delivery machine 32 to adjust a trajectory of the delivered balls as they exit ball delivery machine 32 to enable effective ball delivery to locations at both shorter and longer distances from ball delivery machine 32, to enable varying types of passes (e.g., bounce passes, chest passes, lob passes, or other types of passes), and/or to accommodate for player height.

In some implementations, in operation, controller 94 receives, e.g., from a user on a user device 130, a workout program selection. The workout program includes indications of positions of selected ball delivery locations, ball delivery timing (e.g., tempo) information, a number of balls delivered per location, a type of pass (e.g., chest pass, bounce pass, lob pass, or other type of pass), a selected ball delivery height, and position information of ball delivery machine 32 relative to a visual representation of at least a portion of a basketball court presented by a graphical user interface executed by, e.g., a remote server device. Controller 94 provides control signals to components of the control system of ball delivery machine 32 to deliver balls to selected ball delivery locations according to, e.g., user instructions received via a graphical user interface that presents a visual representation of at least a portion of a basketball court.

The basketball training machine 10 can include and/or be in data communication with one or more sensors, e.g., a sensor 120. Sensor 120 can be an external component in data communication with basketball training machine 10, e.g., a camera of a user device 130. Sensor can be an integrated component of basketball training machine, e.g., a component of a sensing subsystem 124 of the basketball training machine. In some examples, controller 94 can receive data from a sensor 120 of a user device 130, data from a sensor 120 of a sensing subsystem 124, or data from both.

The sensor 120 includes one or more hardware devices capable of receiving input from the environment and converting that input into data accessible by computing elements. For example, the one or more sensors 120 can capture image data, point cloud data, or another format of data capturing two-dimensional (2D) or three-dimensional (3D) information of the training environment, e.g., including a player. The sensor 120 can capture temporal data of the training environment, e.g., to capture shot actions performed by a player over time.

The basketball training machine 10 can include and/or be in data communication with one or more signal sources, e.g., source 122. The source 122 can generate electromagnetic radiation in one or more frequency bands. The electromagnetic radiation can be directed into the training environment, e.g., localized, and the reflections off of surfaces of the training environment, e.g., off of the player's body, the basketball, the floor, etc., can be detectable by sensor 120 to capture information about a training environment surrounding the basketball training machine. For example, the reflections detected by the sensor 120 can be used to extract pose information of a player within the training environment.

Source 122 can be, for example, a radio frequency (RF) source, microwave source, acoustic signal source, laser sources, e.g., for LIDAR, or another non-visible electromagnetic radiation source. For example, a source 122 can be a millimeter (mmWave) frequency source, e.g., between about 3-30 GHz. In another example, a source 122 can be a Wi-Fi frequency source, e.g., between about 2.5-6 GHz. The source 122 can be configured to generate low power signals, e.g., 1000× lower than Wi-Fi signals, or otherwise be compatible with the training environment, e.g., low-harm to humans present in the training environment. A frequency band of the source 122 can be selected based in part on a distance of detection, e.g., distance of player to the basketball training machine, a degree of precision of the detection, a type of sensor 120 performing the detection, compatibility with the environment, or other design considerations.

In some implementations, source 122 can be configured to scan the training environment around the basketball training machine 10. The source 122 can be configured to emit electromagnetic radiation signal in a localized direction and to scan over a range of positions in the training environment, e.g., in an arc sweeping the area surrounding the basketball training machine 10. For example, the source 122 can be a laser source of a LIDAR system coupled to a scanner such that the laser is scanned across the training environment and reflected signal from the training environment is captured by the sensor 120. In another example, the source 122 is a mm Wave source coupled to a scanner, e.g., a mechanical positioning apparatus, such that the mm Wave frequencies are scanned about the training environment and reflected from the training environment is captured by the sensor 120. In such cases, the collected sensor data can include both temporal and spatial resolution.

In some implementations, sensor 120 and source 122 can be integrated into a subsystem, e.g., a sensing subsystem 124 where the sensing subsystem 124 can transmit frequencies into the training environment surrounding the basketball training machine 10. The sensing subsystem can detect a spatial resolution of the reflections as well as a temporal resolution of the detected reflections, e.g., measure intensity, direction, etc., of the reflected signal, to generate point cloud data representing a state of the training environment.

In some implementations, sensing subsystem 124 includes one or more sensors 120 and one or more sources 122. For example, two or more sensors 120 and a source 122. In another example, two or more sensors 120 and two or more sources 122. In another example, two or more sources 122 and a sensor 120. In some instances, the sensors 120 of the sensing subsystem 124 are arranged with respect to the training environment to detect interruptions of a signal emitted by source 122 that is arranged on or in proximity to the basketball training machine 10, e.g., interruptions caused by an object or person located between the source 122 and the sensors 120. In some instances, the source 122 of the sensing subsystem 124 is arranged with respect to the training environment and the one or more sensors 120 are arranged on or in proximity to the basketball training machine 10 to detect interruptions in the emitted signal from the source 122, e.g., interruptions caused by an object or person located between the source 122 and the sensors 120.

In some implementations, the source 122 is a component of the user device, e.g., a cellular phone emitting a source signal. For example, the user device can emit Wi-Fi signal, Bluetooth signal, or another data communication signal. The sensors 120 of the sensing subsystem 124 can be arranged on or in proximity to the basketball training machine 10 and can be configured to detect the emitted signal from the source 122. A strength, quality, or direction of the detected signal emitted by the user device can be detected by the sensors 120 and information of the position of the player and/or objects in the training environment can be inferred.

In some implementations, sensor 120 is camera, e.g., CMOS, CCD, or other device capable of capturing image data and/or video data. The sensor 120 can be configured to capture image data and/or video data in visible wavelengths, infrared wavelengths, or other spectra.

In some implementations, sensor 120 is a detector capable of capturing non-visible electromagnetic radiation signal data, e.g., point cloud data. Sensor 120 can be a detector configured to capture signal in one or more frequency bands, including, for example, millimeter wave (mmWave), radio frequency (RF), Wi-Fi signal bands (2.4 GHz), frequency modulated continuous wave (FMCW) radar (77-81 GHZ), or other frequency bands. For example, sensor 120 can be capable of capturing signal data reflected from (and/or transmitted through) a player's body and capturing pose information of the player.

In some implementations, sensor 120 is an acoustic sensor capable of capturing acoustic signal data. For example, sensor 120 can be an ultrasonic sensor, e.g., a narrow-beam ultrasonic sensor, configured to capture ultrasonic frequencies reflected from a player's body and capturing pose information of the player using the reflected ultrasonic frequencies. An ultrasonic source 122, e.g., an ultrasonic transducer, can be used to detect objects at distance of detection, e.g., up to about 25 meters away from the source. In some examples, an ultrasonic sensor can be integrated with a source, where the transducer of the source functions as a microphone to receive and send the ultrasonic waves, and measures the distance to a target by measuring a time lapse (e.g., time of flight) between sending/receiving the ultrasonic pulse. Ultrasonic waves can have the advantage of being able to detect clear or transparent objects, e.g., liquids, and can be stable over a large array of surface finish, material, and color. Ultrasonic-based sensing subsystems can be insensitive to ambient lighting conditions. In some examples, a narrow beam ultrasonic sensor can be used to provide increased resolution within a distance range useful for the training environment. In another example, sensor 120 can be configured to capture low-level acoustic signals reflected from and/or transmitted through a player's body and capturing pose information of the player using the low-level acoustic signals.

In some implementations, the basketball training machine 10 includes distance sensors such as a light detection and ranging (LIDAR) sensor, a time-of-flight sensor, etc. The data, images, and/or video captured by the image sensor may also be processed to reduce the noise and/or vibration in the signal using. In some implementations, sensor 120 collects pre-data which the basketball training system 96 can process to extract biometric data and/or pose information for the player. For example, the sensor 120 collects point cloud data from which the basketball training system may pre-process, e.g., filter, and extract biometric data for the player. In some implementations, the system can pre-process the sensor data to reduce signal-to-noise, filter, or otherwise clean up the sensor data prior to extracting the action data. The system can pre-process the data by flattening 3D sensor data to a 2D space. In some implementations, sensor 120 is a radar sensor, e.g., mmWave sensor, collecting radar data. Sensor 120 can be integrated with or in data communication with additional processing components, e.g., hardware accelerators, digital signal processors, or the like, which process the radar data to generate further processed data forms. For example, radar data can be analog-to-digital (ADC) data output from the sensor 120, e.g., frames where each frame is a representation of collected chirps for the period of time of the frame (framerate). The radar data can be further processed to produce refined data, e.g., point cloud heat maps, using low-level radar processing. A further refinement of the data can include a first high-level radar processing to produce target tracking and motion/object decisions, e.g., using target information extraction. Another further refinement of the data can include a second high-level radar processing to produce target classification, e.g., using classification information and/or micro-Doppler effects. The additional processing components can generate, from the refined radar data, a visualization including scene interpretation from which form training process described herein can proceed.

In some implementations, the basketball training system 96 includes two or more sensors 120 of a same or different type. In some examples, the basketball training system 96 includes at least one camera and at least one non-visible electromagnetic radiation sensor. In such examples, the sensors 120 can collect multi-modal data input, e.g., data originating from two or more different types of sensors. For example, image data and point cloud data, which can be used collectively by the system to generate form training information, e.g., as described in further detail below.

The sensor 120 can be mounted such that the field of view of the sensor 120 is capable of capturing images of a player when the player receives a ball and takes a shot. In some cases, this can involve two or more sensors integrated into the ball delivery system 14, and/or one or more sensors physically separate from the rest of the ball delivery system 14 (e.g. in addition to or in the alternative to an integrated sensor). For example, an image sensor is an integrated sensor 120 of a user device 130, e.g., cellular phone with a camera, in data communication with the basketball training machine 10. In some examples, a sensor 120 is a component of a sensing subsystem 124 of the basketball training system 96.

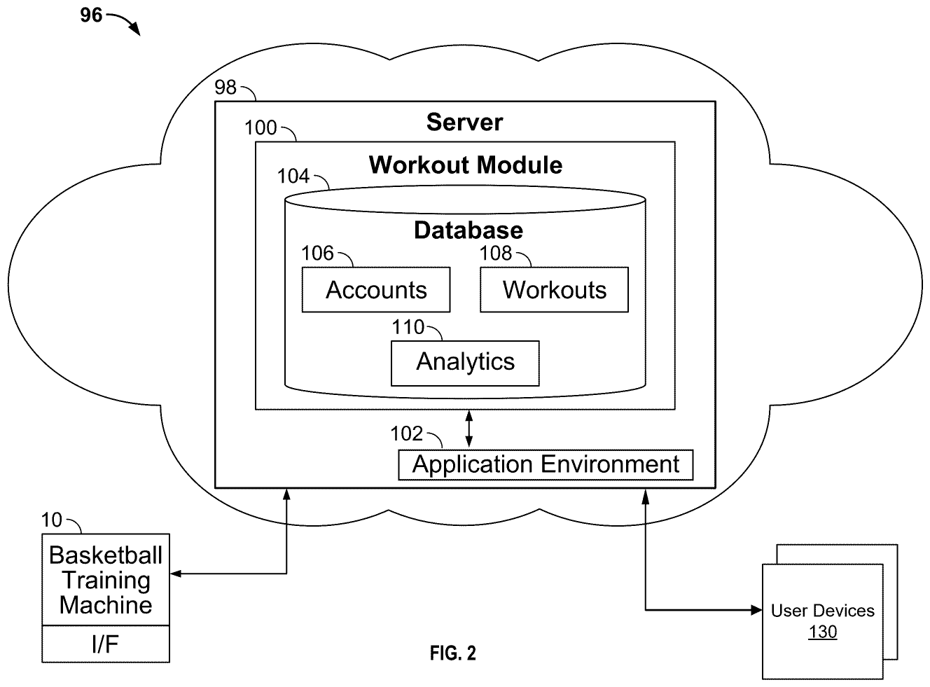

FIG. 2 is a block diagram of basketball training system 96 that includes basketball training machine 10 communicatively coupled with server 98 that executes workout module 100 to generate a workout program executed by basketball training machine 10. As illustrated in FIG. 2, server 98 includes workout module 100 and application environment 102. Workout module 100 includes database 104, which includes accounts 106, workouts 108, and analytics 110.

Server 98, as illustrated in FIG. 2, can be a cloud-based or otherwise remote server computer including one or more processors and computer-readable memory encoded with instructions that, when executed by the one or more processors, cause server 98 to execute workout module 100 and application environment 102 according to techniques described herein. Examples of server 98 include, but are not limited to, mainframe computers, desktop computers, laptop computers, or other computing devices capable of implementing workout module 100 and managing operation of (e.g., serving of) application environment 102. In some examples, rather than a single server device, server 98 may be implemented as a server system including multiple interconnected server computers that distribute functionality attributed herein to server 98 among the multiple server computers. For instance, in certain examples, server 98 can be implemented as a server system including a first server computer that executes workout module 100 and a second server computer that executes (e.g., serves) application environment 102. Similarly, while database 104 is illustrated as included in workout module 100 of server 98, database 104 can include any one or more databases that may be local to server 98 or distributed among any one or more server devices operatively connected to server 98.

Workouts 108 store workout programs that include machine workout instructions that are executed by basketball training machine 10 to deliver basketballs to selected ball delivery locations as well as player workout instructions that represent player activity (e.g., player movement, skill development activities such as dribbling or other ball handling maneuvers, exercise activities such as pushups or sit-ups, or other player activity). Workouts 108 are associated with attributes, such as a workout skill level, workout intensity level, workout time, workout type (e.g., offensive skills development, long range shooting development, short range shooting development, free throw shooting development, agility development, strength development, ball handling development, physical conditioning development, or other workout type), or other attributes.

Workout programs stored at workouts 108 can be associated with any one or more accounts and/or account groups stored in database 104 at accounts 106. For instance, a particular workout program can be associated with (e.g., assigned to) an account group corresponding to a team, and therefore also associated with each individual user account that is a member of the team account group through the hierarchical relationship between the parent team account group and the child user accounts. Workout programs stored at workouts 108 can be associated with a single account of accounts 106 or multiple accounts of accounts 106. As such, workout programs can be generated and stored at workouts 108 of database 104 and utilized by a single user account or shared between multiple user accounts or account groups.

Analytics 110 of database 104 store analytics data (e.g., statistics) associated with any one or more accounts stored at accounts 106 and/or workout programs stored at workouts 108. Examples of analytics data include form training data (e.g., action profiles), shooting percentage data, a number of attempted shots, a usage time of basketball training machine 10, user heart rate data during any one or more workout programs (e.g., sensed by a heart rate monitor or other physical monitoring device worn by a player during a workout program), shooting percentage relative to heart rate, movement, position on the basketball court, or other analytics data. Analytics data stored at analytics 110 of database 104 can be associated with a workout program, such that each user account that executes a particular workout program contributes to shared analytics corresponding to the executed workout program. In general, analytics 110 can store any statistical or other analytical data that corresponds to user accounts, user account groups (e.g., team account groups), and workout programs to enable comparison of performance between user accounts, between user accounts and benchmark performance criteria, between time-separated performances of a single user account (or account group), or other comparisons. As such, analytics data stored at analytics 110 can enable a coach, player, or other user to track performance of a single player or group of players over time, to compare performances between players or groups of players, and to track progress of skill development and conditioning of players or groups of players. For example, analytics 110 can enable a coach, player, or other user to track user form compared to an ideal form and/or a self-consistent form for multiple different action shots, e.g., three-point shot, free-throw, dynamic shots, static shots, dribbling, etc., over time and between a same session or two or more different sessions.

As illustrated in FIG. 2, server 98 can execute an application environment 102, for example, through a web browser accessible on a user device 130 or in a local application located on the user device 130. User devices 130 can be, for example, tablet computers, mobile phones (including smartphones), laptop computers, wearable devices such as smartwatches, or other computing devices that can communicate with server 98 to interface with workout module 100. Application environment 102 is communicatively coupled with workout module 100 to provide a user interface that enables user interaction with workout module 100 to create and select workout programs and view analytics data stored at database 104, as is further described below.

Basketball training machine 10 is communicatively coupled with server 98 to access application environment 102 via any one or more wired or wireless communication networks, such as a cellular communication network, local area network (LAN), wide area network (WAN) such as the Internet, wireless LAN (WLAN), or other type of communication network. In operation, a player utilizes user device 130 (e.g., a smartphone) to execute application environment 102 through an application (e.g., an app) that interfaces with workout module 100 or to access application environment 102 via a web browser that presents a graphical user interface managed by workout module 100. The graphical user interface presents a login screen that enables the player to provide account login information, such as username and passcode. Workout module 100 accesses the account stored in accounts 106 associated with the login information (or enables the player to create a new account) and presents the user with graphical control elements to either select a workout program stored at workouts 108 of database 104 or create a new workout program, as is further described below.

Basketball training machine 10, as illustrated in FIG. 2, includes an interface (illustrated as I/F), such as console 58, a touchscreen interface, a keyboard and/or mouse interface, or other type of interface to enable user interaction with application environment 102 to create a workout program and/or select a workout program stored at workouts 108 for execution by basketball training machine 10.

In operation, a user selects, through the graphical user interface of application environment 102, a workout program stored at workouts 108 and/or create a new workout program via the interface provided by application environment 102 and managed by workout module 100. Server 98 transmits the selected or created workout program to basketball training machine 10. The workout program includes both machine workout instructions for execution by basketball training machine 10 and player workout instructions representing player activity during workout program. Basketball training machine 10 executes the machine workout instructions by delivering basketballs to identified ball delivery locations at a selected tempo (i.e., relative timing) and tracking made and missed shots via shots made sensor 26. Basketball training machine 10 presents, through a graphical user interface of the application environment 102, player instructions for review prior to execution of the workout program and, in certain examples, presents the player instructions during execution of the workout program via a display, speakers, or other output device. Results of the workout program corresponding to made and missed shots, duration of one or more portions of the workout program, or other analytics data can be transmitted by basketball training machine 10 to workout module 100 of server 98. Accordingly, system 96 enables a user to select one or more workout programs stored at workouts 108 of database 104, create a new workout program that can optionally be stored in workouts 108, and execute the workout program to enable effective training for the player.

A user of the basketball training machine 10, e.g., a player, coach, or other interested part, can interact with the basketball training machine 10 through a graphical user interface (GUI) of the application environment 102, that receives user input and provides feedback to the user through the graphical user interface. As described in further details below, the graphical user interface can be presented in an application environment on a user device 130, e.g., a player's mobile device, or in display of a console 58 of the basketball training machine 10. A user can interact with the basketball training machine 10 through the graphical user interface, for example, to define a workout program that includes selected ball delivery locations desired by a user and to receive feedback related to a workout program in progress and/or completed, e.g., form training.

The basketball training system 96 presents, through the graphical user interface, graphical control elements that enable the user to interact with the basketball training machine 10, e.g., to select a workout. The graphical user interface can be managed by a server device, communicatively coupled with a controller 94 of the basketball training machine 10 or a separate computing device, e.g., a user device 130, to receive the workout program including machine workout instructions executed by basketball training machine 10 and player workout instructions presented to the user.

As such, the basketball training system 96, through to the graphical user interface, enables a user (e.g., a player, coach, administrator, training expert, or other user) to define a workout program via the graphical user interface that is communicated to ball delivery machine 32 of the basketball training machine 10 for execution and, in certain examples, enables selection of desired ball delivery locations that are not limited by indications of predetermined ball delivery locations. In this way, the basketball training machine 10 enables the generation of workout programs by differing users via one or more computing devices that are communicatively coupled with a server device that manages operation of the graphical user interface.

In some implementations, the basketball training system 96 presents, through the graphical user interface, a visual representation of at least a portion of a basketball court representing predetermined ball delivery locations on the basketball court, such as visual markings, buttons, lights, or other physical or graphically-rendered indications of predetermined ball delivery (or shot) locations. In such examples, the graphical user interface enables a greater range of ball delivery locations and player movement that can help to simulate game-like scenarios and increase an effectiveness of training.

The basketball training system 96 can receive, through the graphical user interface, inputs from a user (e.g., gesture input at a touch-sensitive and/or presence-sensitive device, input from a mouse, keyboard, voice command, or other input) relative to a presented visual representation of the basketball court that identify the selected ball delivery locations.

Though described with reference to FIG. 2 as a basketball training system 96 in data communication with a basketball training machine 10, in some implementations, the basketball training system 96 provides the form training processes described in this specification without using some or all of the functionality of the basketball training machine 10. In some examples, the basketball training system 96 can collect captured sensor data using a standalone sensing subsystem 124 and/or from a sensor 120 of a user device 130, where the captured sensor data includes the player performing the shot action. In such cases, the system can provide guidance to the user to collect the sensor data of the player performing the shot action using a basketball hoop, e.g., a standard play basketball hoop or another configuration not in data communication with the basketball training system 96. The guidance can include positioning of the camera of the user device 130 or components of the sensing subsystem 124 with respect to a player's position and a receiving basketball hoop. The system can receive the collected data from the user device 130 and/or the sensing subsystem 124 and process the collected sensor data at server 98 to proceed with the form training process, as described herein.

### Form Training Process

As described with reference to FIGS. 1 and 2, the basketball training system 96 can be used to perform form training for a player. Form training includes determining consistency of a player's form performing an action shot (e.g., dribbling, dynamic shot, static shot, or other movement) with respect to an ideal form and/or a self-consistent form. A self-consistent form includes the player's own historical form data, in other words, how their form performing the action shot varies over time within a single workout program or multiple workout programs.

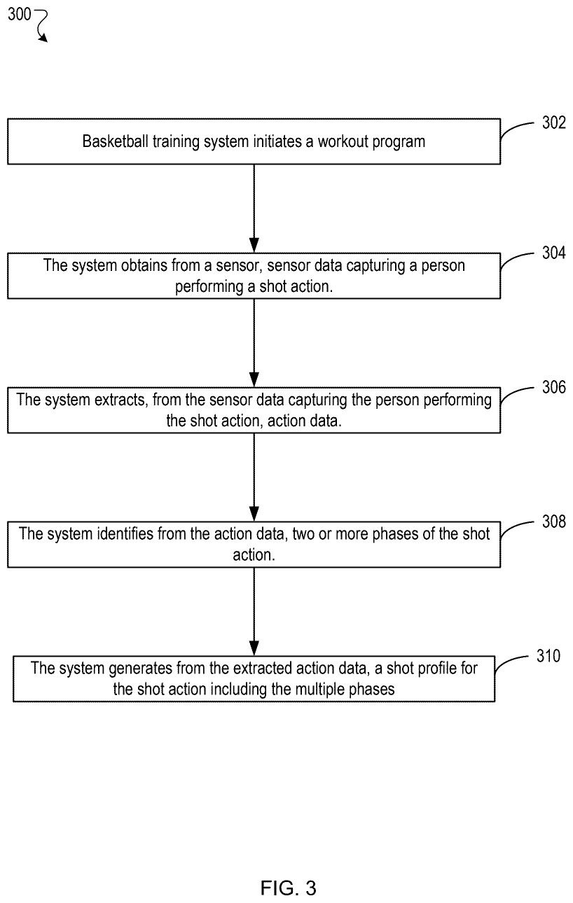

FIG. 3 is a flow diagram of an example process 300 for collecting and indexing pose information for a player using a basketball training system. Basketball training system initiates (302) a workout program. The system can initiate the program in response to receiving a selected workout program from a user, e.g., the player or their trainer. The basketball training system can provide an audio and/or visual representation of the workout program to the user, e.g., through the graphical user interface of the application environment, to provide guidance before, during, and after the workout program is run. The basketball training system provides to various components of the basketball training machine 10 control signals to cause the basketball training machine 10 to enact the workout program, e.g., specifying pass locations, number of passes, etc.

The system obtains (304), from one or more sensors, sensor data capturing a person performing a shot action. For example, the basketball training machine 10 can pass the ball to the player, the player can take a shot, etc. As this occurs, the sensor 120 can capture sensor data of the player. As described above, the sensor can be, for example, a camera of the user's device 130 having a field of view of the user as the user is performing the workout program. In some examples, the sensor can be configured to capture sensor data in a frequency band of the electromagnetic spectrum outside the visible frequency band. The sensor 120 can be an integrated component of the basketball training machine 10 or a component of a user device 130 in data communication with the basketball training machine 10. At times, multiple sensors 120 can be used to capture sensor data of the shot action simultaneously or sequentially. The multiple sensors 120 can be the same or different types of sensors, e.g., a camera and a mmWave sensor, each capturing respective sensor data. A position of the field of view of the sensor can be specific to a target angle, e.g., face-on, side-view, or another angle, of the user's form with respect to the basketball training machine 10.

In some implementations, the system obtains the sensor data, e.g., video data including the multiple frames, by capturing sensor data including the person performing the shot action two or more times. The system can capture sensor data simultaneously from two or more different angles of a same shot action (or set of shot actions), e.g., using the same or different sensors 120. Alternatively, or additionally, the system can capture video data sequentially of the user performing the shot action from two or more different angles.

In some implementations, the system obtains the sensor data from two or more different types of sensors, e.g., multi-modal sensor data, from the same or different angles of the same shot action (or set of shot actions). For example, the system can obtain video data and point cloud data from respective sensors of the shot action (or set of shot actions) captured by respective sensors.

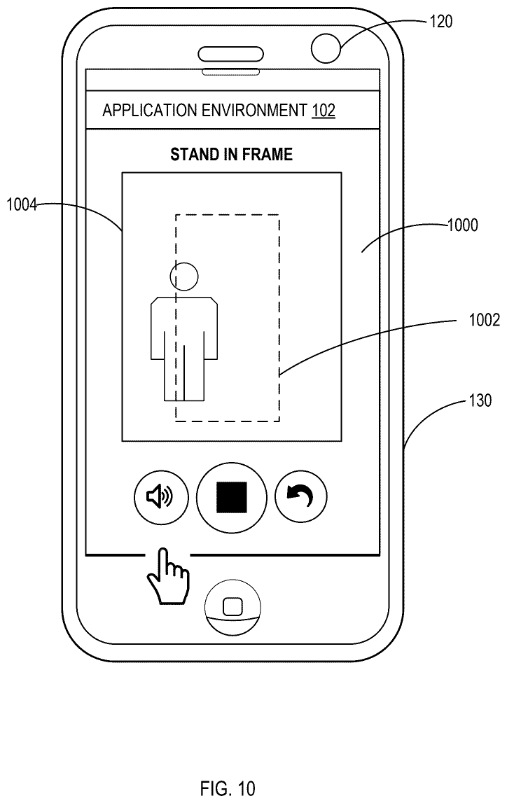

In some implementations, the basketball training system 96 assists, through the graphical user interface, a user in positioning the one or more image sensors, e.g., a camera of a user device 130, with respect to the basketball training machine 10 to capture video data of the user performing a selected workout program, e.g., for form training. For example, as depicted in FIG. 10, the graphical user interface 1000 can present a visual indicator 1002, e.g., a highlighted region, outlined region, arrows, or other indicators, within a display 1004 of a real-time capture of image data from the camera 120 of the user device 130 to guide the user on where to position the user device 130 in order to capture one or more angles of the user performing the workout program. For example, the graphical user interface can guide the user to position the camera of the user device to capture a face-on view, a side-view, or an angled view of the user when the user is positioned with respect to the basketball training machine 10 during a workout program.

In some implementations, the basketball training system 96 assists, through the graphical user interface, a user in positioning themselves with respect to one or more sensors, e.g., sensors of the sensing subsystem 124 of the basketball training machine 96. For example, the graphical user interface can present visual cues for the user to follow to align themselves within the field of view of the integrated sensor of the basketball training machine.

In some implementations, obtaining the sensor data, e.g., video, includes instructing, by the system and through the graphical user interface, e.g., as depicted in FIG. 10, the user to move a location of the image sensor (e.g., the camera of the user device) from a first video data capture location to a second video data capture location, e.g., to capture video data of the user performing the shot action from multiple angles.

In some implementations, obtaining the sensor data, e.g., point cloud data, includes instructing, by the system and through visual cues to the user, the user to move to a different location, e.g., from a first sensor data capture location to a second sensor data capture location, with respect to the sensor to capture sensor data of the user performing the shot action from multiple angles.

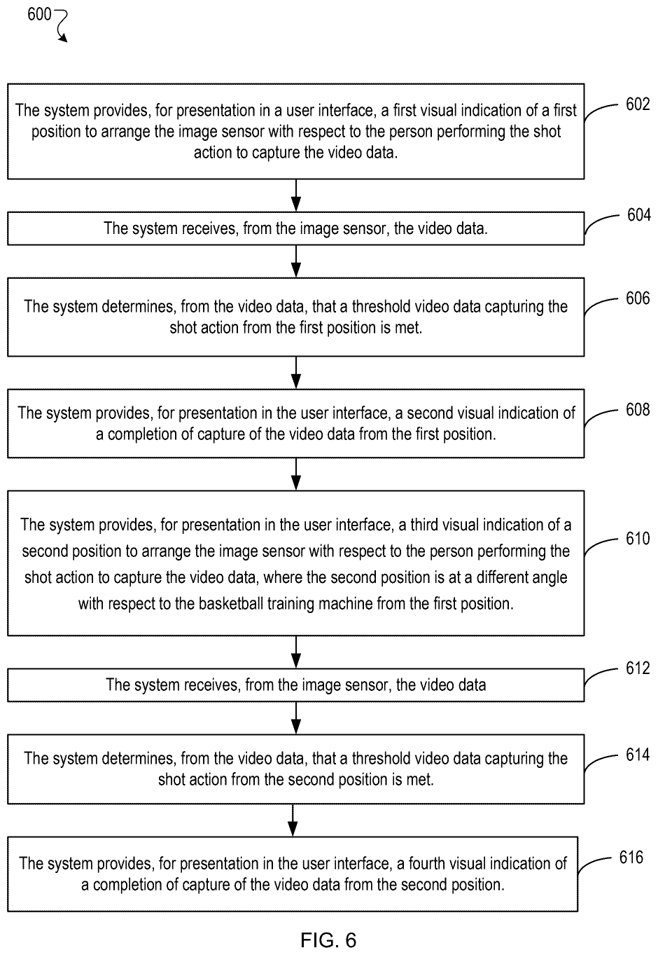

FIG. 6 is a flow diagram of an example process 600 for a basketball training system. For convenience, the process 600 will be described as being performed by a system of one or more computers, located in one or more locations, and programmed appropriately in accordance with this specification. For example, a basketball training system, e.g., the basketball training system 96 of FIG. 2, appropriately programmed, can perform the process 600. Additionally, although the example process 600 is described with respect to an image sensor and captured video data, the processes described with reference to FIG. 6 can be performed by one or more sensors configured to capture respective sensor data, where at least one of the one or more sensors is configured to capture sensor data in a non-visible wavelength spectrum, e.g., mmWave, Wi-Fi, or the like.

The system provides (602), for presentation in a user interface, a first visual indication of a first position to arrange the image sensor with respect to the person performing the shot action to capture the video data. For example, the system can guide the user to position the user device in a first location to capture a first angle of the user's form performing the shot action(s), e.g., face-on, for a first set of shot actions, and subsequently the system can guide the user to re-position the user device in a second location to capture a second angle of the user's form performing the shot action(s), e.g., side-view for a second set of shot actions. Some or all of the guidance can be represented visually in the graphical user interface to assist the user in aligning the field of view of the image sensor to capture the correct angle of the user's form. For example, the graphical user interface can include a target box, arrows, or other indicators, highlighting a location of where a user's body should be located during the capture of the video data, e.g., as depicted in FIG. 10.

The system receives (604), from the image sensor, the video data, and determines (606), from the video data, that a threshold video data capturing the shot action from the first position is met. For example, the system can determine that a threshold number of shot actions are captured from the first position. A threshold for the number of shots can depend, in part, on an amount of biometric data points extractable from the location, e.g., if some of the biometric data points are obscured or not discernable by the pose information model.

In some implementations, the system receives sensor data from two or more sensors, e.g., multi-modal sensor data, and determines, from the sensor data that a threshold sensor data capturing the shot action from the first position is met. For example, the multi-modal sensor data can include video data captured by an image sensor and point cloud data captured by a mmWave detector.

The system provides (608), for presentation in the user interface, a second visual indication of a completion of capture of the video data from the first position. The system can provide visual, audio, and/or haptic feedback to the user to indicate that the portion of the workout program is completed and to move on to the next step or conclude the workout program.

The system provides (610), for presentation in the user interface, a third visual indication of a second position to arrange the image sensor with respect to the person performing the shot action to capture the video data, where the second position is at a different angle with respect to the basketball training machine from the first position.

The system receives (612), from the image sensor, the video data and determines (614), from the video data, that a threshold video data capturing the shot action from the second position is met. For example, the system can determine that a threshold number of shot actions are captured from the second position. In some instances, a threshold number of shot actions can be different between the first position and the second position. For example, the system may require fewer shots performed from a face-on view than shots performed from a side-view of the user. A threshold for the number of shots can depend, in part, on an amount of biometric data points extractable from the location, e.g., if some of the biometric data points are obscured or not discernable by the pose information model.

In some implementations, the system receives sensor data from two or more sensors, e.g., multi-modal sensor data, and determines, from the sensor data that a threshold sensor data capturing the shot action from the second position is met. For example, the multi-modal sensor data can include video data captured by an image sensor and point cloud data captured by a mm Wave detector.

The system provides (616), for presentation in the user interface, a fourth visual indication of a completion of capture of the video data from the second position. The system can provide visual, audio, and/or haptic feedback to the user to indicate that the portion of the workout program is completed and to move on to the next step or conclude the workout program.

In some implementations, the system can provide, to a trained machine learning model, video data capturing the shot action from the first position, and obtain as output, from the trained machine learning model, extrapolated synthetic video data representing the shot action from a second, virtual position.

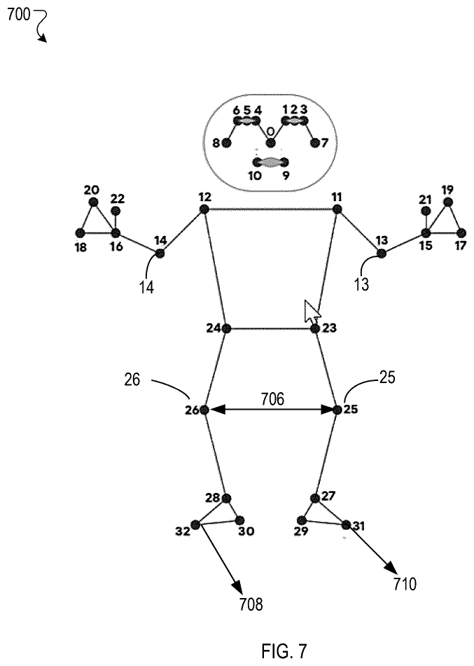

Referring back to FIG. 3, the system extracts (306), from the sensor data capturing the person performing the shot action, action data. For example, the system extracts the action data from frames of captured video data including the person performing the shot action. In another example, the system extracts the action data from multi-modal sensor data. The action data includes biomechanical pose information including multiple points, e.g., coordinates in two-dimensional or three-dimensional space, tracking the biomechanics of the shot action. The multiple points track respective locations of biometric data points corresponding to the person appearing in the sensor data and tracking the shot action. For example, as depicted in FIG. 7, a computer vision pose detection model is used to translate sensor data capturing a user's body into a biomechanical form 700 of points, e.g., elbow points 13, 14, detecting and tracking the user's limb positions and movements thereof before, during, and after the shot action. The pose detection model can further be used to define relative locations between points, e.g., distance 706 between knee points 25 and 26. The pose detection model can be used to define directions, e.g., vectors, of movement, for example, vectors 708, 710 indicating directions of movement of the feet of the form.

In some implementations, extracting biomechanical pose information includes resolving the most likely arrangement of a pose rig including a data structure to represent joints and rigid elements of the human body. A solver can find, for example, the angle values for each joint most likely (e.g., with the lowest error value) to produce an outline of a human matching an outline found of the player. It will be appreciated that other processes for determining pose information can be used, including the use of machine-learning trained classifiers. Action data includes timing information for the shot action with the pose information. For example, at each frame, a data object to represent the pose can be stored in a datastore and indexed based on a time value. The time value may be a 24-hour clock representing time of day, may be a time value measuring time elapsed since the start of the workout, or may be a frame number.

In some implementations, the system can access action data generated by other biomechanical tracking sensors, e.g., wearable technology, that tracks body movements, to enrich the action data extracted from the video data. The system can use the additional action data from biomechanical tracking sensors to detect, log, and reinforce positions of body points of the user over time.

In some implementations, the system integrates action data including object detection data for one or more objects. In other words, the action data further includes tracking respective locations of an object appearing in the sensor data, e.g., the basketball. Tracking the respective locations of the object can include tracking the object relative to the biometric data points corresponding to the person in the sensor data and/or relative to another (e.g., stationary) frame of references. In some examples, the system can track the position of the ball relative to the biometric points corresponding to the dynamic position of the user's hands and/or track the position of the ball with a static frame of reference, e.g., the camera's world coordinates. The object position can be tracked through space and time to extract one or more object metrics. For example, an object metric includes an angle the object as it travels in an arc and approaches the goal (e.g., the basketball net of the basketball training machine 10). In another example, an object metric includes a spin rate of the object, e.g., rotations of the ball in revolutions per unit time. In another example, an object metric includes a contact/no contact with boundaries of the goal, e.g., the rim of the hoop, as the object passes through the boundaries. In another example, an object metric includes a dribble speed.

In some implementations, the system enriches the action data with shot completion feedback, e.g., make/miss information. In some cases, the basketball training system may be equipped with a rim-sensor that is able to observe where, relative to the rim, a made shot passes. Since the rim of a hoop is larger than the ball, the ball can pass through the hoop and be counted as “made” when passing through an area away from the center of the hoop. This information is useful because, in many cases, most shots should be aimed to pass through the center of the hoop. However, some shot types (e.g., layups) may be better targeted in other areas (e.g., near the back of the hoop). These sensors can include, but are not limited to, any of the sensors described in this specification, contacting sensors such as vibration sensors, or pressure switches attached to or positioned within the hoop. These sensors can include, but are not limited to, non-contacting sensors such as vision sensors or time of flight sensors. In still other examples, the sensors used to collect the rim information may include data other than video such as LIDAR data.

In some implementations, the system integrates shot completion feedback with the action data. For example, using the techniques described in this document, shot completion feedback for each shot in a workout can be created and indexed. Similarly, the ball-path information of each shot action can be indexed so that each shot has an associated form data-object and also an associated ball-path data object. In one example, a “distance from center” calculation can be performed to find how far a particular ball path is from the center of the rim. In another example, a “distance from ideal” calculation can be performed to find how far a particular ball path is from an ideal location in the hoop, which may or may not be the center of the hoop.

With this information, the basketball training system 96 can draw correlations between the shot profile of the player's form and shot outcome. For example, the basketball training system may determine that a particular type of form breakdown (e.g., insufficient elevation) is correlated with made shots that nevertheless are consistently left of center. In another example for another player, the system may determine that similar form breakdown is not correlated with any such inaccuracies in ball placement.

For example, workout information involving the ball can be logged by the system and used to enrich the action data collected from the sensor data, e.g., shot completion feedback. A ball passing machine can pass a ball to the player and determine makes, misses, etc. for each shot, or other drill metrics can be observed using the computer vision (e.g., was the shot taken with sufficient elevation) or other sensors (e.g., as a ball detected passing through the hoop).

The system identifies (308), from the action data, two or more phases of the shot action. The shot action is deconstructed into multiple phases, e.g., two or more phases, each phase of the shot action represented by a subset of the biometric data points that track a timing and movement of the respective phase of the shot action. Each phase of the two or more phases can be defined by coordinate points and vectors for the points through a duration of the phase of the action shot. In some implementations, the phases can be defined by manual labeling, automated labeling, or a combination thereof, where identification of coordinate points and vectors for the points (defining the movement of the points through space) for the phase through the duration of each phase can be performed in an automatic, semi-automatic, or manual process. For example, human experts can label a sequence of shots actions to identify the durations of each of the phases and identify the relevant points used to define the phase. In another example, one or more trained machine learning models can receive a sequence of shot actions as input and provide, as output, predictions of coordinate points and vectors for the points corresponding to each phase of the shot action.

In some implementations, the phases of the shot profile include position, shot pocket, lift, shot path, follow through, and landing. Each of the phases correspond to a particular movement within the shot action where the system can use a subset of the action data extracted from the captured sensor data of the shot action to define the start, duration, and end of the phase. Phases can be defined using respective subsets of points of the action data, distances/relative locations between selected subset of points of the action data, coordinated movements of particular subsets of points of the action data, angular relationship between limbs as defined by subsets of points of the action data, or any combination thereof. Phases can be defined by timing of absolute and/or relative movements of subsets of points of the action data. Each phase can be defined by a different measure of a respective subset of the action data.

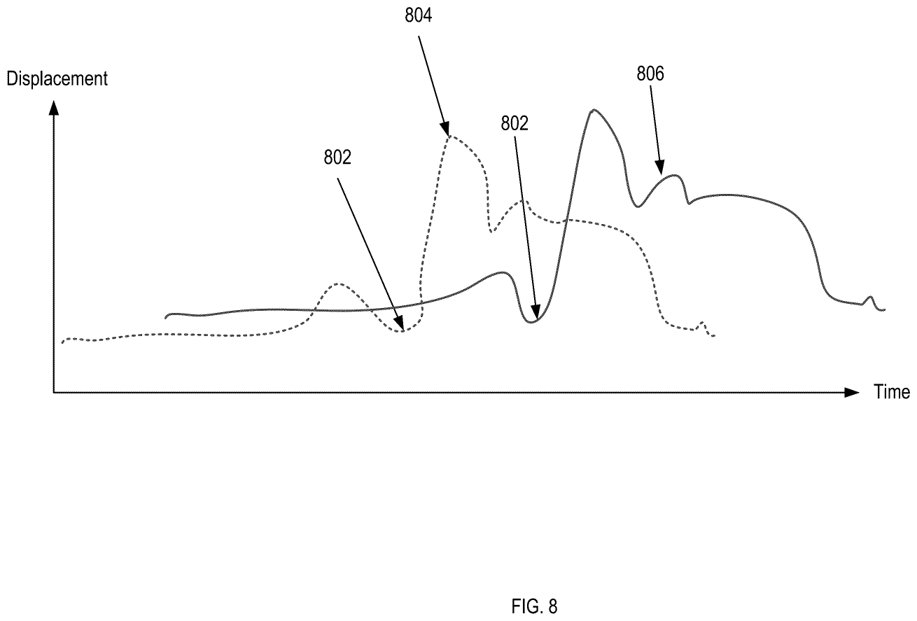

In one example, the shot pocket phase is defined when an identified subset of points of the biometric data are located at a lowest overall point, e.g., at point 802 depicted in FIG. 8. The shot pocket location can be defined based on the locations of a subset of points, e.g., left and right shoulders, left and right hips, and left and right knees.

An exemplary calculation of the shot pocket follows, though more/fewer points and/or different subset of points are contemplated. Referring to the points depicted in FIG. 8, where each point corresponding to a feature of the body form is referred to as “point N” or “X-N” where “N” is the numbered element. A distance between two points is referred to as “D_N1_N2” where N1 and N2 are two points between which the distance is calculated. The center of the shot pocket runs directly through the center of the eye of the player depending on their handedness. Therefore, the coordinate for the “X” center of the shot pocket (SP_x) corresponds to point 5, e.g., “X_5,” for right-handed shooters, and point 2, e.g., “X_2,” for left-handed shooter. An added offset, (SP_xos) can be used for convenience, where SP_xos is a function of the width of the shot pocket using the coefficient SP_xc=0 nominally. If SP_xc was 0.1 for example, then the shot pocket would be moved 10% of the width of the shot pocket to the right for a right-handed shooter, and 10% of width to the LEFT for a left-handed shooter, in other words:

SP_xos=SP_w*SP_xc

For a right-handed player the equation would be:

SP_x=X_5+SP_xos

For a left-handed player the equation would be:

SP_x=X_2−SP_xos

The coordinate for the “Y” center of the shot pocket SP_y is defined as the average “Y” coordinate of the hips plus an offset SP_yos where the offset is a function of the height of the shot pocket using the coefficient SP_yc=−0.3 nominally:

SP_yos=SP_h*SP_yc

SP_y=(Y_23+Y_24)/2+SP_yos

The height of the shot pocket (SP_h) is defined as the average distance between the knees and the hips times the shot pocket height coefficient SP_hc. SP_hc=1.0. A distance between the hip and knees “D_24_26” and “D_23_25” is therefore:

D_24_26=SQRT((X_24−X_26)\hat{ }2+(Y_24−Y_26)\hat{ }2)

D_23_25=SQRT((X_23−X_25)\hat{ }2+(Y_23−Y_25)\hat{ }2)

SP_h=SP_hc*(D24_26+D23_25)/2

If at any point a value is not available, only D_24_26 or D_23_25 will be used depending on which value is missing The width of the shot pocket (SP_w is defined as the distance between the hips times the shot pocket width coefficient SP_wc, where the SP_wc=0.8). A distance between points 23 and 24 (hip distance), e.g., “D_23_24,” is therefore:

D_23_24=SQRT((X_23−X_24)\hat{ }2+(Y_23−Y_24)\hat{ }2)

A width of the shot pocket, SP_w is then:

SP_w=SP_wc*D_23_24

In another example, the position phase includes a position of the player before the ball is caught and can be defined based in part on, for example, the position of the hip positions extracted from the biometric data.

In another example, a lift phase is defined at a start of the basketball shot, e.g., at an end point of the shot pocket phase, and ends at a release point of the ball, e.g., as defined by an angular relationship between a subset of points.

In another example, a shot path phase is defined at the start of the y-coordinate dip (e.g., the shot pocket) and an ends when the elbow is at a 90 degrees, or when the elbow passes the shoulder points.

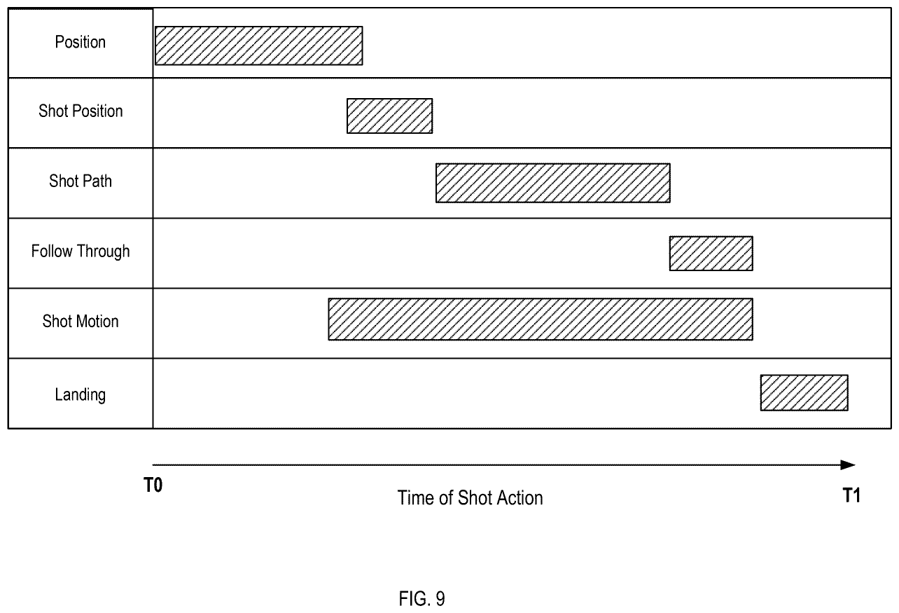

In some implementations, at least one phase of the two or more phases overlaps in timing with at least one other phase of the two or more phases during the shot action. FIG. 9 depicts a block diagram of an example of phases of the shot profile over the timing of the shot action. As depicted, a shot motion phase overlaps with at least a portion of position, shot position, shot path, and follow through phases.

In some implementations, different shot actions can be deconstructed into different sets of phases. For example, a shot action including a “dribble then shoot” movement can have a distinct set of phases in a corresponding shot profile than a shot action include a “catch then shoot” movement.

Referring back to FIG. 3, the system generates (310), from the extracted action data, a shot profile for the shot action including the multiple phases. FIG. 8 depicts to examples of shot profiles 804 and 806 corresponding to two shot actions with respect to a displacement of the action data points, e.g., along a y-axis, and captured over time T. Although similar in appearance, shot profiles 804 and 806 deviate from each over both in the displacement tracked along the coordinate axis as well as the timing of the executed movements. Each of the shot profiles exhibits a respective reference point 802, by which the system can align the shot profiles 804 and 806 on a same plot and perform the aggregate analysis described herein.

Each phase relates to a shot action-specific movement within the shot action that relates the pose data to a specific phase metrics. In some implementations, the system further evaluates phase metrics for each of the phases of the shot profile to determine a deviation of the user's form for the phase against a target form. A target form can be, for example, an ideal form (as defined by the basketball training system, experts in the field, a trainer or coach, or another authority on form) and/or a self-consistent form defining an aggregated historical form for the particular user over a previous amount of time.

Phase metrics can be defined to mathematically define the relationships between the biomechanics of the shot action represented by the phases of the shot profile and the outcome of the shot (e.g., make, miss, miss bias, etc.). The phase metrics can be used to perform comparisons between the user's shot profile and an ideal shot profile, and can be used to measure consistency and/or fidelity of the shot action and make performance recommendations. Each phase can have associated phase metrics to define the aspects of the phase. At times, a phase metric can span two or more phases. For example, a phase metric can define the start and finish of a player's shot path and measure an efficiency of movement within the path. In another example, a phase metric can measure a player's ability to keep the basketball within a defined area, e.g., during a shot pocket phase, of the shot action and generates a corresponding score indicating a fidelity to the area. In another example, a phase metric can measure a time of the player in the air, e.g., during a jump, during a shot action. In another example, a phase metric can measure a degree to which the ball is maintained a vertical up/down only trajectory. In another example, a phase metric can measure a fluidity and coordination of the player's body swaying during the shot, e.g., the smoother the motion, the higher the score. In another example, a phase metric can measure a speed of a wrist flick when the player releases the ball during the shot action. In another example, a phase metric can measure a deviation between the distance that the player's heels are apart when their feet leave the ground, and the distance between their heels when they touch back down on the ground. In another example, a phase metric can measure an average release time for a set of shots. In another example, a phase metric can determine fastest and slowest release times of a set of shots. In another example, a phase metric measures a duration of the player's hand(s) in an upward position after shooting. In another example, a phase metric measures a speed performing a sequences of actions of the action shot, e.g., speed of catching of the ball, dribbling, and shooting. In another example, a phase metric measures an agility and precision of the player's footwork leading up to the shot. In another example, a phase metric measures a consistency of a shooting rhythm, e.g., timing. In another example, a phase metric measures a length and control of an extension of the player's shooting arm. In another example, a phase metric measures a precision and steadiness of the player's eye alignment with the target, e.g., the hoop. In another example, a phase metric measures overall synchronization and coordination of all body parts during the single shot. In another example, a phase metric measures a fidelity of the expected body part movements at an optimal time as compared to other body parts. In another example, a phase metric quantifies an aggregate score of a player's body stability and balance using the pose coordinates during the action shot. In another example, a phase metric measures a movement and speed of the ball from when the player catches the ball until the player releases the ball.

In some implementations, one or more phase metrics can be assessed in combination and/or in comparison to each other to generate global phase metrics. Phase metrics can also be generated based on evaluation of multiple shot profiles in aggregate. For example, a phase metric can measure an accuracy of footwork, jump height/landing position, shot arc, shot completion, or the like, over an aggregate of multiple shot actions taken by the player, e.g., during a workout program.

In some implementations, the basketball training system can leverage data collected by one or more sensors configured to capture non-visible light signal data, e.g., frequencies of the electromagnetic radiation spectrum outside the visible light spectrum, in addition to or instead of video and/or image data collected by an image sensor. For example, the basketball training system can leverage multi-modal sensor data collected by different types of sensors, e.g., at least one sensor configured to capture non-visible light signal data and at least one image sensor configured to capture video and/or image data. The multi-modal data can be enriched sensor data which includes additional insight, e.g., can include more information about the action than the video data alone.

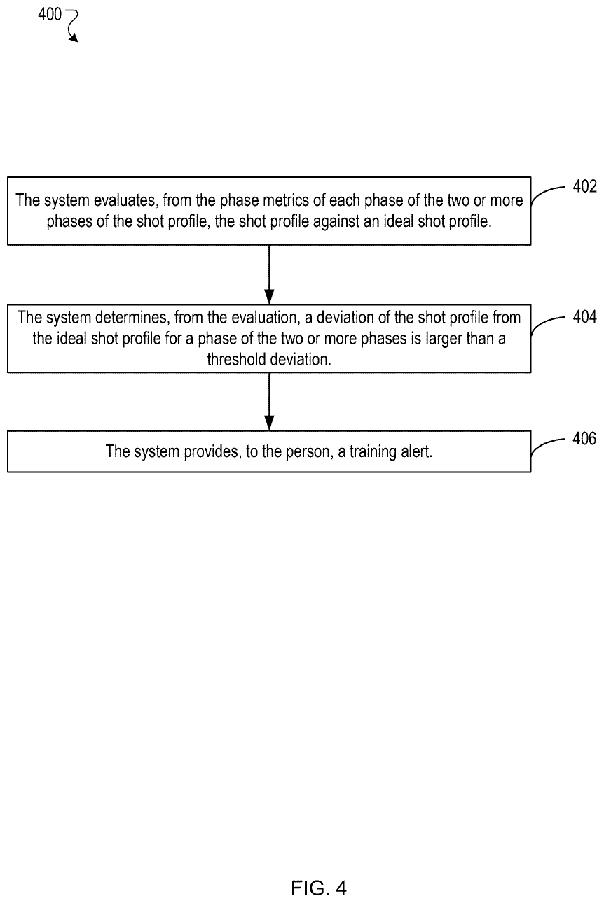

FIG. 4 is a flow diagram of an example process 400 for the basketball training system. For convenience, the process 400 will be described as being performed by a system of one or more computers, located in one or more locations, and programmed appropriately in accordance with this specification. For example, a basketball training system, e.g., the basketball training system 96 of FIG. 2, appropriately programmed, can perform the process 400.

The system evaluates (402), from the phase metrics of each phase of the two or more phases of the shot profile, the shot profile against an ideal shot profile. As described above, the system can compare the shot profile to an ideal shot profile and/or self-consistent shot profile stored in database 104 for the particular shot action and/or the set of shot actions for the workout program. The comparison can include a comparison of timing and/or movement of each of the phases, where each phase is defined by the respective phase metrics. For example, a library of “ideal” or “exemplary” form data can be stored in computer memory. For each shot, a corresponding target form would specify the best form possible for that given shot. This can be defined in terms of form as described above, but also or instead in terms of phase and/or phase metric, depending on how the library is constructed and maintained.

With this comparison, the system can determine a measure of the quality of the shot according the different phases and corresponding phase metrics in addition to the make or miss of the shot. For example, a player may make a number of shots in spite of the fact that they place their hands on the improper portion of the ball or take an idiosyncratic hop before taking their shot. Similarly, a player may execute proper form on a high-difficulty shot (e.g., long 3-point shot from the top of the court) that nevertheless misses. With this comparison, the system can determine a player's process (i.e., their form) instead of their outcome (i.e., make or miss). As will be appreciated, good form can be more predictive of future success in a particular drill than good outcomes—a player with good form who just misses high-difficulty shots is more likely to progress than a player with inconsistent and wild form but who got lucky on a few shots.

The system determines (404), from the evaluation, a deviation of the shot profile from the ideal shot profile for a phase of the two or more phases is larger than a threshold deviation. In some implementations, determining the deviation of the shot profile from the ideal shot profile for the phase includes determining a type of deviation of multiple types of deviations for the phase.

In some implementations, the system can determine an overall shot score for the shot action. The system can determine the overall shot score as a combined score of each of the respective scores for the phases of the shot profile. At times, the overall shot score can be a weighted value, e.g., where one phase is weighted more than another, different phase. At times, the overall shot score can be weighted, based in part on the shot completion feedback, e.g., whether the shot was made, missed, or based on the miss bias. The overall score can be report, for example, on a scale of 0 to 1 or 1 to 100 can include the various variances available upon request to help understand what contributed to the consistency score, e.g., broken down by phases and/or phase metrics.

In some implementations, the system uses the phase metrics to assess a consistency of the player's shot action. Consistency can be defined as how consistent a player is in summation (i.e., total shots taken into consideration) as well as piecemeal (i.e., each phase of the shot). This will allow players to focus on areas in need of more attention. The system can measure consistency mathematically in various ways as described herein including the calculation of “standard deviation” of a player form over a series of shots. Consistency can be calculated as a numerical representation (e.g., a consistency score) and depicted, in the graphical user interface, through charts and graphs, as well as a visualization of multiple shots “overlayed” on top of each other to shows similarities and differences from one shot to another.

In addition to measuring form in a controlled environment (“static form” measurement), the system allows players to shoot on the move (i.e., moving left, right, forward, or back (“fadeaways”)) and measure their form (and consistency of form) dynamically. In addition to measurement of dynamic form, the system can categorize shots, e.g., automatically, semi-automatically, or by a user manually, with specific labels in order to allow for robust analysis of the player's form in different contexts/dynamics. For example, form training can be applied to categories such as “moving left, moving right, fadeaway, driving toward the basket, off the dribble, off a pass, relaxed, catch-and-shoot”.

The system provides (406), to the person, a training alert. A training alert can be provided, by the system, visual display by way of a screen, audio display by way of a speaker, haptic display by way of a tactile element such a worn device with a vibration element, etc.

The basketball training system can provide the user with feedback that the player can incorporate while performing the workout. In one example, a player taking the low-probability 3-point shots can take a shot that ends up as a miss. For this shot, the system can determine that the user's form is good in spite of missing the shot. In response, the basketball training system can play a message through a speaker such as “Good form! You'll get it soon.” Similarly, if the player then takes another shot with poor form, the real time feedback can tell the user what part of their form was off “Keep your eyes on the hoop next time” can be played if, for example, it is determined that the head angle was out of compliance on that shot.

In some cases, this feedback and other feedback described in this document can be provided in a visual format that compares the recorded form to an ideal or exemplar form. In one instance, a shot with form representative of a player's performance in a drill can be presented as a video contemporaneous with the video of the exemplar. In one case, the player form, e.g., a skeletal representation or body form representation, can be overlaid in the video with partial transparency over video of a professional or expert player taking the same kind of shot. In one case, the player's video can be shown next to the video of the expert.

In some implementations, providing the training alert by the system further includes determining, by the system and for the phase having a deviation larger than the threshold deviation, a training regimen specific to the phase and different than a training regimen for each other phase of the two or more phases, and providing the training alert. The system can determine the training regimen, e.g., workout, drills, etc., specific to the phase by determining the training regimen of a repository of training regimens that is specific to the type of deviation for the phase. For example, a first set of drills can be selected responsive to a timing deviation of the phase (e.g., too slow/fast, pace is off), a second set of drills can be selected responsive to a movement deviation of the phase (e.g., arms are in improper position relative to hips).

In some implementations, the system provides the training alert by providing, for presentation in a user interface, a visual representation indicating the phase of the two or more phases for which the deviation of the shot profile from the ideal shot profile is larger than the threshold deviation. In one example, the visual representation can include a listing of the different phases where each phase is color-coded, highlighted, or otherwise identified distinctly depending on the determined deviation of the shot profile from the ideal shot profile for the phase.

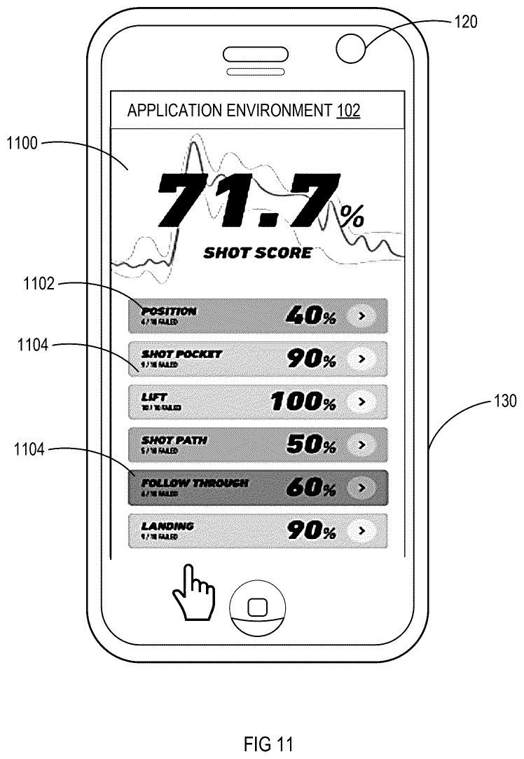

For example, as depicted in the graphical user interface 1100 depicted FIG. 11, each of the phases, e.g., phases 1102, 1104, 1106, displayed in the graphical user interface is coded with a different color/texture/shade to indicate “within threshold,” “borderline with respect to the threshold,” or “outside threshold,” e.g., using color indicators of green, yellow, and red. Additionally, each of the phases is displayed with a percentage value indicating a measure of alignment of the shot profile with the ideal shot profile, e.g., accuracy, consistency, etc.

In some implementations, the system provides, for presentation in the user interface, a selectable control operable to initiate the training regimen specific to correct a detected deviation for the phase.

In some implementations, the system further evaluates the shot profile for the shot action using one or more trained machine learning models. The system can use one or more trained machine learning models to receive, as input, shot action data and provide, as output, a prediction of a form deviation for the shot profile of the shot action. For example, the system can provide shot action data as input to one or more trained machine learning models, e.g., one model trained on shot pocket data, one model trained on follow-through data, etc. Each of the trained machine learning models can be trained using a respective, specific data set corresponding to collected data across multiple different players all performing the phase. As such, the trained machine learning models can be robust to different player sizes, variation in technique, deviations in video capture/resolution/camera position, and the like.

In some implementations, the machine learning models are trained using shot action data including or derived from multi-modal sensor data as input. For example, the training data can include video data and point cloud data of the shot action.

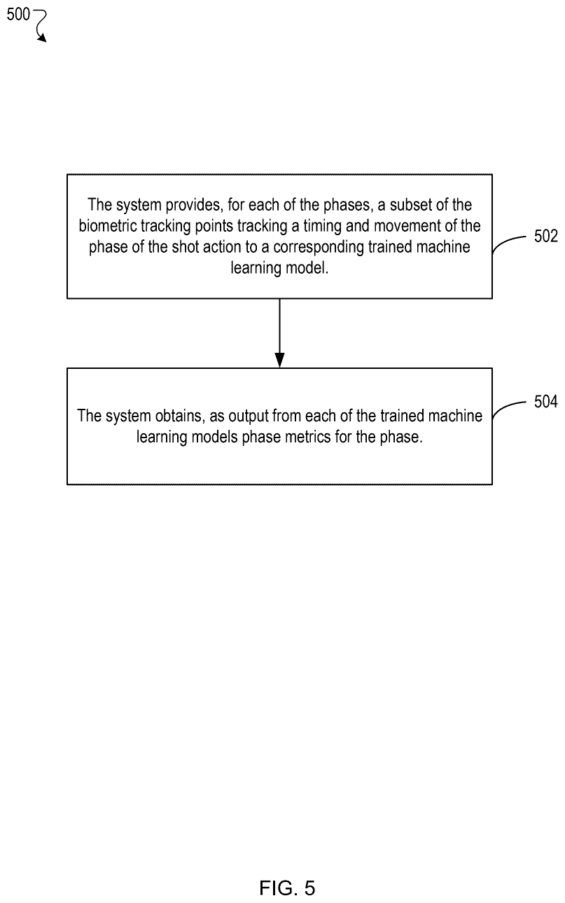

FIG. 5 is a flow diagram of an example process 500 for the basketball training system. For convenience, the process 500 will be described as being performed by a system of one or more computers, located in one or more locations, and programmed appropriately in accordance with this specification. For example, a basketball training system, e.g., the basketball training system 96 of FIG. 2, appropriately programmed, can perform the process 500. The system provides (502), for each of the phases, a subset of the biometric tracking points tracking a timing and movement of the phase of the shot action to a corresponding trained machine learning model. Alternatively, or additionally, the system can provide the sensor data capturing the phase of the shot action to the trained machine learning model as input.

The system obtains (504) as output from each of the trained machine learning models phase metrics for the phase. The phase metrics, as described above, can include a predictions of deviation(s) of the user's form from an ideal form and/or self-consistent form for the phase of the shot profile. Phase metrics can include a prediction including a confidence score of the alignment of the user's form for the phase with the ideal form and/or self-consistent form.

In some implementations, the system collects sensor data capturing the user performing the shot action two or more times, e.g., using one or more sensors, and aggregates the shot profiles corresponding to respective shot actions, e.g., in a sequence of actions performed during a workout program. This can allow the system to perform an aggregated analysis over multiple performed action shots and reduce any error introduced by accidental mistakes or transient issues.

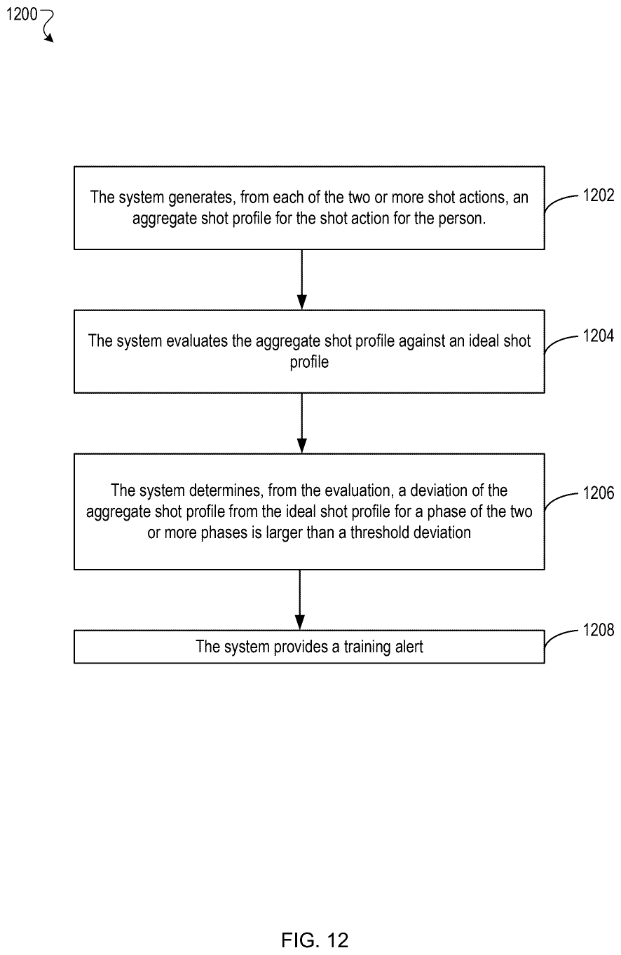

FIG. 12 is a flow diagram of an example process 1200 of the basketball training system. For convenience, the process 1200 will be described as being performed by a system of one or more computers, located in one or more locations, and programmed appropriately in accordance with this specification. For example, a basketball training system, e.g., the basketball training system 96 of FIG. 2, appropriately programmed, can perform the process 1200. The system generates (1202), from each of the two or more shot actions, an aggregate, e.g., average, shot profile for the shot action for the person. The system can generate a respective shot profile for each of the performed shot actions and perform an analysis of the user's form for the shot action over the multiple performed shot actions. In other words, the shot profile for each shot is combined with the shot profile for every other shot.

In some implementations, system identifies a reference point of the shot profile, e.g., a shot pocket location corresponding to a lowest coordinate biometric point along the y-axis and aligns each of the shot profiles using the respective reference points of the shot profiles. The system evaluates (1204) the aggregate shot profile against an ideal shot profile, determines (1206), from the evaluation, a deviation of the aggregate shot profile from the ideal shot profile for a phase of the two or more phases is larger than a threshold deviation and provides (1208) a training alert.

In some implementations, the data of the corresponding shot profiles for each shot action of the multiple performed shot actions are aggregated. The aggregated data can be further processed e.g., to filter outliers, erroneous data, and reduce noise, to refine the data, to remove outliers. The system can determine a mean, median, mode, or other form of averaging or normalization of the data of each of the shot profiles to generate an aggregated shot profile.

The subject matter and the actions and operations described in this specification can be implemented in digital electronic circuitry, in tangibly-embodied computer software or firmware, in computer hardware, including the structures disclosed in this specification and their structural equivalents, or in combinations of one or more of them. The subject matter and the actions and operations described in this specification can be implemented as or in one or more computer programs, e.g., one or more modules of computer program instructions, encoded on a computer program carrier, for execution by, or to control the operation of, data processing apparatus. The carrier can be a tangible non-transitory computer storage medium. Alternatively or in addition, the carrier can be an artificially-generated propagated signal, e.g., a machine-generated electrical, optical, or electromagnetic signal, that is generated to encode information for transmission to suitable receiver apparatus for execution by a data processing apparatus. The computer storage medium can be or be part of a machine-readable storage device, a machine-readable storage substrate, a random or serial access memory device, or a combination of one or more of them. A computer storage medium is not a propagated signal.

The term “data processing apparatus” encompasses all kinds of apparatus, devices, and machines for processing data, including by way of example a programmable processor, a computer, or multiple processors or computers. Data processing apparatus can include special-purpose logic circuitry, e.g., an FPGA (field programmable gate array), an ASIC (application-specific integrated circuit), or a GPU (graphics processing unit). The apparatus can also include, in addition to hardware, code that creates an execution environment for computer programs, e.g., code that constitutes processor firmware, a protocol stack, a database management system, an operating system, or a combination of one or more of them.

A computer program can be written in any form of programming language, including compiled or interpreted languages, or declarative or procedural languages; and it can be deployed in any form, including as a stand-alone program, e.g., as an app, or as a software module, component, engine, subroutine, or other unit suitable for executing in a computing environment, which environment may include one or more computers interconnected by a data communication network in one or more locations.

A computer program may, but need not, correspond to a file in a file system. A computer program can be stored in a portion of a file that holds other programs or data, e.g., one or more scripts stored in a markup language document, in a single file dedicated to the program in question, or in multiple coordinated files, e.g., files that store one or more modules, sub-programs, or portions of code.

The processes and logic flows described in this specification can be performed by one or more computers executing one or more computer programs to perform operations by operating on input data and generating output. The processes and logic flows can also be performed by special-purpose logic circuitry, e.g., an FPGA, an ASIC, or a GPU, or by a combination of special-purpose logic circuitry and one or more programmed computers.

Computers suitable for the execution of a computer program can be based on general or special-purpose microprocessors or microcontrollers or a combination of them, or any other kind of central processing unit. Generally, a central processing unit will receive instructions and data from a read-only memory or a random access memory or both. The essential elements of a computer are a central processing unit for executing instructions and one or more memory devices for storing instructions and data. The central processing unit and the memory can be supplemented by, or incorporated in, special-purpose logic circuitry.

Generally, a computer will also include, or be operatively coupled to, one or more mass storage devices, and be configured to receive data from or transfer data to the mass storage devices. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

To provide for interaction with a user, the subject matter described in this specification can be implemented on one or more computers having, or configured to communicate with, a display device, e.g., a LCD (liquid crystal display) monitor, or a virtual-reality (VR) or augmented-reality (AR) display, for displaying information to the user, and an input device by which the user can provide input to the computer, e.g., a keyboard and a pointing device, e.g., a mouse, a trackball or touchpad. Other kinds of devices can be used to provide for interaction with a user as well; for example, feedback and responses provided to the user can be any form of sensory feedback, e.g., visual, auditory, speech, or tactile feedback or responses; and input from the user can be received in any form, including acoustic, speech, tactile, or eye tracking input, including touch motion or gestures, or kinetic motion or gestures or orientation motion or gestures. In addition, a computer can interact with a user by sending documents to and receiving documents from a device that is used by the user; for example, by sending web pages to a web browser on a user's device in response to requests received from the web browser, or by interacting with an app running on a user device, e.g., on a smartphone or electronic tablet. Also, a computer can interact with a user by sending text messages or other forms of message to a personal device, e.g., a smartphone that is running a messaging application, and receiving responsive messages from the user in return.

This specification uses the term “configured to” in connection with systems, apparatus, and computer program components. That a system of one or more computers is configured to perform particular operations or actions means that the system has installed on it software, firmware, hardware, or a combination of them that in operation cause the system to perform the operations or actions. That one or more computer programs is configured to perform particular operations or actions means that the one or more programs include instructions that, when executed by data processing apparatus, cause the apparatus to perform the operations or actions. That special-purpose logic circuitry is configured to perform particular operations or actions means that the circuitry has electronic logic that performs the operations or actions.

While this specification contains many specific implementation details, these should not be construed as limitations on the scope of what is being claimed, which is defined by the claims themselves, but rather as descriptions of features that may be specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially be claimed as such, one or more features from a claimed combination can in some cases be excised from the combination, and the claim may be directed to a subcombination or variation of a subcombination.

Similarly, while operations are depicted in the drawings and recited in the claims in a particular order, this by itself should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the implementations described above should not be understood as requiring such separation in all implementations, and it should be understood that the described program components and systems can generally be integrated together in a single software product or packaged into multiple software products.

Particular implementations of the subject matter have been described. Other implementations are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A basketball training system comprising:
a basketball training machine;
a sensor; and
one or more processors coupled to one or more computer-readable storage media having instructions stored thereon which, when executed by the one or more processors, cause the one or more processors to perform operations comprising:
obtaining, from the sensor, sensor data including a person performing a shot action;
extracting, from the sensor data, action data comprising a plurality of points tracking the shot action, wherein the plurality of points track respective locations of biometric data points corresponding to the person in the sensor data and tracking the shot action;
identifying, from the action data, two or more phases of the shot action, wherein each of the two or more phases of the shot action include a proper subset of the plurality of points tracking a timing and movement of the phase of the shot action; and
generating, from the extracted action data, a shot profile for the shot action comprising the two or more phases.

2. The system of claim 1, further comprising evaluating the shot profile for the shot action, wherein the evaluating comprises:
providing, for each of the two or more phases of the shot action and to a corresponding trained machine learning model of a plurality of trained machine learning models, the proper subset of the plurality of points tracking the timing and movement of the phase of the shot action; and
obtaining, for each of the two or more phases of the shot action, phase metrics for the phase.

3. The system of claim 2, wherein the plurality of trained machine learning models are each trained on a respective, different data set corresponding to a phase of the two or more phases of a shot profile for the action shot.

4. The system of claim 2, further comprising:
evaluating, from the phase metrics of each phase of the two or more phases of the shot profile, the shot profile against an ideal shot profile;
determining, from the evaluation, a deviation of the shot profile from the ideal shot profile for a phase of the two or more phases is larger than a threshold deviation; and
providing, to the person, a training alert.

5. The system of claim 4, wherein providing the training alert further comprises:
determining, for the phase having a deviation larger than the threshold deviation, a training regimen specific to the phase and different than a training regimen for each other phase of the two or more phases; and
providing, to the person, the training alert.

6. The system of claim 5, wherein determining the deviation of the shot profile from the ideal shot profile for the phase comprises determining, a type of deviation of a plurality of types of deviations for the phase, and
wherein determining the training regimen specific to the phase comprises determining the training regimen of a plurality of training regimens that is specific to the type of deviation of the plurality of types deviations for the phase.

7. The system of claim 5, wherein providing the training alert comprises:
providing, for presentation in a user interface, a visual representation indicating the phase of the two or more phases for which the deviation of the shot profile from the ideal shot profile is larger than the threshold deviation; and
providing, for presentation in the user interface, a selectable control operable to initiate the training regimen specific to the phase.

8. The system of claim 1, wherein obtaining the sensor data comprises obtaining the sensor data including the person performing the shot action two or more times, and
wherein generating the shot profile comprises generating a respective shot profile for each of the two or more performed shot actions by the person.

9. The system of claim 8, wherein obtaining the sensor data further comprises:
identifying, for each of the respective shot profiles, a corresponding reference point of the shot profile; and
aligning the shot profiles for the two or more performed shot actions using the respective reference points of the shot profiles.

10. The system of claim 9, further comprising:
generating, from each of the two or more shot actions, an average shot profile for the shot action for the person; and
evaluating, from the average shot profile against an ideal shot profile;
determining, from the evaluation, a deviation of the average shot profile from the ideal shot profile for a phase of the two or more phases is larger than a threshold deviation; and
providing, to the person, a training alert.

11. The system of claim 1, wherein the action data comprising the plurality of points tracking the shot action further comprises tracking respective locations of an object captured by in the sensor data, and
wherein tracking the respective locations of the object comprises tracking the object relative to the biometric data points corresponding to the person in the sensor data.

12. The system of claim 1, wherein each phase of the two or more phases is defined by coordinate points and vectors for the points through a duration of the phase of the action shot.

13. The system of claim 1, wherein the two or more phases of the shot action comprise position, shot pocket, lift, shot path, follow through, and landing.

14. The system of claim 1, where at least one phase of the two or more phases overlaps in timing with at least one other phase of the two or more phases during the shot action.

15. The system of claim 1, wherein the sensor comprises an image sensor, and
wherein the sensor data comprises video data including a plurality of frames including the person performing the shot action.

16. The system of claim 15, wherein obtaining, from the image sensor, the video data comprising the plurality of frames including the person performing the shot action comprises:
providing, for presentation in a user interface, a first visual indication of a first position to arrange the image sensor with respect to the person performing the shot action to capture the video data;
receiving, from the image sensor, the video data;
determining, from the video data, that a threshold video data capturing the shot action from the first position is met; and
providing, for presentation in the user interface, a second visual indication of a completion of capture of the video data from the first position.

17. The system of claim 16, wherein obtaining, from the image sensor, the video data comprising the plurality of frames including the person performing the shot action further comprises:
providing, for presentation in the user interface, a third visual indication of a second position to arrange the image sensor with respect to the person performing the shot action to capture the video data, wherein the second position is at a different angle with respect to the basketball training machine from the first position;
receiving, from the image sensor, the video data;
determining, from the video data, that a threshold video data capturing the shot action from the second position is met; and
providing, for presentation in the user interface, a fourth visual indication of a completion of capture of the video data from the second position.

18. The system of claim 17, wherein the first position is a side view of the person performing the shot action, and wherein the second position is a face-on view of the person performing the shot action.

19. The system of claim 17, wherein determining, from the video data, that the threshold video data capturing the shot action from the first position and the second position is met comprises:
determining that a threshold number of shot actions are captured in the plurality of frames of the video data from the respective first position and the second position.

20. The system of claim 19, where the threshold number of shot actions is different between the first position and the second position.

21. The system of claim 16, wherein obtaining, from the sensor, the sensor data including the person performing the shot action further comprises:
providing, to a trained machine learning model, the sensor data capturing the shot action from the first position; and
obtaining, from the trained machine learning model, extrapolated synthetic sensor data representing the shot action from a second, virtual position.

22. The system of claim 1, wherein the shot action comprises the action of
receiving, by the person and from a delivery device of the basketball training machine, a basketball; and
shooting, by the person and to a receiving hoop of the basketball training machine, the basketball.

23. The system of claim 1, further comprising:
obtaining, from the basketball training machine and for the shot action, shot completion feedback; and
generating, from the shot profile and the shot completion feedback; an enriched shot profile for the shot action.

24. The system of claim 1, wherein the sensor is configured to capture sensor data in a non-visible electromagnetic spectrum frequency band.

25. The system of claim 24, wherein the sensor is configured to capture sensor data in one of radio frequency bands, microwave frequency bands, acoustic frequency bands, and infrared frequency bands.

26. The system of claim 24, wherein the sensor data comprises point cloud data.

27. The system of claim 24, wherein the sensor is configured to capture sensor data in an ultrasonic frequency band.

28. The system of claim 27, wherein the sensor comprises a narrow-beam ultrasonic sensor.

29. A sports training system comprising:
a sport training machine;
a sensor; and
one or more processors coupled to one or more computer-readable storage media having instructions stored thereon which, when executed by the one or more processors, cause the one or more processors to perform operations comprising:
obtaining, from the sensor, sensor data including a person performing a player movement;
extracting, from the sensor data, action data comprising a plurality of points tracking the player movement, wherein the plurality of points track respective locations of biometric data points corresponding to the person appearing in the plurality of frames and tracking the player movement;
identifying, from the action data, two or more phases of the player movement, wherein each of the two or more phases of the player movement include a proper subset of the plurality of points tracking a timing and movement of the phase of the player movement; and
generating, from the extracted action data, a movement profile for the player movement comprising the two or more phases.

30. The sports training system of claim 29, wherein the sensor comprises a first sensor to obtain first second data and further comprising a second sensor,
wherein the operations further comprise:
obtaining, from the second sensor, second sensor data including the person performing the player movement;
extracting, from the second sensor data, second action data comprising a second plurality of points tracking the player movement, wherein the second plurality of points track respective locations of biometric data points corresponding to the person captured in the second sensor data and tracking the player movement.

31. The sports training system of claim 30, wherein the operations further comprise:
identifying, from the action data and the second action data, two or more phases of the player movement, wherein each of the two or more phases of the player movement include a proper subset selected from the plurality of points and the second plurality of points tracking a timing and movement of the phase of the player movement; and
generating, from the extracted action data and second action data, the movement profile for the player movement comprising the two or more phases.

32. The sports training system of claim 31, wherein the first sensor comprises an image sensor and is configured to capture video data and the second sensor is configured to capture the second sensor data in one of radio frequency bands, microwave frequency bands, acoustic frequency bands, and infrared frequency bands.

33. The sports training system of claim 32, wherein the proper subset is selected from both of the plurality of points and the second plurality of points.

34. A sports training method comprising:
providing, by an emitter, a source signal comprising a non-visible wavelength;
collecting, from a sensor and from reflections of the source signal, sensor data including a person performing a player movement;
extracting, from the sensor data, action data comprising a plurality of points tracking the player movement, wherein the plurality of points track respective locations of biometric data points corresponding to the person appearing in the plurality of frames and tracking the player movement;
identifying, from the action data, two or more phases of the player movement, wherein each of the two or more phases of the player movement include a proper subset of the plurality of points tracking a timing and movement of the phase of the player movement; and
generating, from the extracted action data, a movement profile for the player movement comprising the two or more phases.
